(19) United States
(12) Patent Application Publication
(10) Pub. No.: US 2019/0325988 A1
(43) Pub. Date: Oct. 24, 2019

(54) METHOD AND SYSTEM FOR RAPID GENETIC ANALYSIS

(71) Applicant: RADY CHILDREN'S HOSPITAL RESEARCH CENTER, San Diego, CA (US)

(72) Inventors: Stephen Kingsmore, San Diego, CA (US); Narayanan Veeraraghavan, Poway, CA (US); Michelle Marie Clark, Vista, CA (US)

(21) Appl. No.: 16/388,614

(22) Filed: Apr. 18, 2019

Related U.S. Application Data
(60) Provisional application No. 62/659,495, filed on Apr. 18, 2018.

(51) Int. Cl. G16B 25/10; C12Q 1/6883

(52) U.S. Cl. CPC G16B 25/10; C12Q 2600/172; C12Q 1/6883

(57) ABSTRACT

The present disclosure provides a method for genetic analysis disease diagnoses as well as a system for implementing such analysis.

### CROSS REFERENCE TO RELATED APPLICATION(S)

This application claims the benefit under 35 USC § 119(e) to U.S. Application Ser. No. 62/659,495 filed Apr. 18, 2018. The disclosure of each of the prior application(s) is considered part of and is incorporated by reference in the disclosure of this application.

### BACKGROUND OF THE INVENTION

### Field of the Invention

The invention relates generally to genetic analysis and more specifically to a method and system for rapid characterization of genetic disease.

### Background Information

Genetic diseases are the leading cause of infant mortality in the US, particularly among the approximate 15% of infants admitted to neonatal, pediatric and cardiovascular intensive care units (ICUs) (1-11). As disease progression in infants is rapid, etiologic diagnosis must be equally fast to inform interventions that can lessen suffering, morbidity and mortality (12, 13). Unfortunately, this is rarely the case. More than 13,000 genetic diseases are known (14, 15). Their presentations often overlap in seriously ill infants and are typically abridged with respect to classical descriptions (14, 15). Standard genomic sequencing takes weeks to return results, which is too slow to guide inpatient management. Rapid whole genome sequencing (rWGS) provides faster diagnosis, enabling precision medicine interventions in time to decrease the morbidity and mortality of infants with genetic diseases (12, 13). Furthermore, in genetic diseases with uniformly dismal prognosis, rapid diagnosis facilitates end-of-life care decisions that can alleviate suffering and aid the grieving process. Clinical studies are starting to substantiate the diagnostic and clinical utility and cost effectiveness of rapid genomic sequencing in seriously ill infants in ICUs, with reported rates of diagnosis of 42-57%, changes in medical management in 30-72%, and altered outcomes in 24-34% of cases (12, 14, 16-30). This evidence has led to calls for accelerated implementation in national healthcare systems as the new standard of care (31-33). The National Health Service of the United Kingdom (UK), for example, will offer rapid whole genome sequencing as part of care for all seriously ill children from 2019 (34). The clinical utility of rapid whole genome sequencing is also being studied in older children and adults in medical and cardiac ICUs. The major impediments to universal implementation in ICUs are absence of reimbursement outside the UK, lack of knowledge of genomic medicine by pediatricians, and the high capital and labor intensity of current, clinical, rapid genome sequencing and interpretation.

Diagnosis by rapid genome sequencing in 26 hours in a research setting has been previously reported (16, 17). In the clinical studies reported to date, however, the fastest genetic diagnosis by genomic sequencing was 37 hours, mean time-to-diagnosis was sixteen days, and largest cohort comprised only sixty three patients (8, 16-30). The small cohort size and longer time-to-diagnosis in those clinical studies substantiate the limitations of current methods of rapid genomic sequencing. More advanced methods are needed for clinical diagnosis of genetic diseases with automated provisional diagnosis as described herein.

### SUMMARY OF THE INVENTION

The present invention provides a method and autonomous system for conducting genetic analysis. The invention provides for rapid diagnosis of genetic disease.

Accordingly, in one embodiment the invention provides a method for conducting genetic analysis. The method includes:

a) determining a phenome of a subject from an electronic medical record (EMR), wherein the phenome includes a plurality of clinical phenotypes extracted from the EMR;

b) translating the clinical phenotypes into standardized vocabulary or vocabularies;

c) generating a first list of potential differential diagnoses of the subject;

d) performing genetic sequencing of a DNA sample from the subject;

e) determining genetic variants of the DNA;

f) analyzing the results of (c) and (e) to generate a second list of potential differential diagnoses of the subject, the second list being rank ordered; and

g) generating a report including results of the analysis of (f).

In embodiments, the method further includes generating the EMR for the subject prior to determining the phenome of the subject. In embodiments, translating the clinical phenotypes into standardized vocabulary is performed by extraction of phenotypes by clinical natural language processing (CNLP) and then translation into one or more standardized vocabularies. In embodiments, genetic sequencing includes rWGS, rapid whole exome sequencing (rWES), or rapid gene panel sequencing.

In another embodiment, the invention provides a method for performing genetic analysis in a plurality of subjects. The method includes:

a) generating a plurality of EMRs for a plurality of subjects;

b) determining a plurality of phenomes of the plurality of subjects from the EMRs using natural language processing, wherein the phenomes each having a plurality of clinical phenotypes extracted from each of the EMRs;

c) storing on a non-transitory memory the plurality of EMRs, the plurality of phenomes, and the plurality of clinical phenotypes to generate a searchable database; and

d) utilizing the database to screen for a disease or disorder in a new subject or to update a diagnosis of one of the plurality of subjects.

In another embodiment, the invention provides a system for performing the method of the invention. The system includes a controller having at least one processor and non-transitory memory. The controller is configured to perform one or more of the processes of the method as described herein.

### BRIEF DESCRIPTION OF THE DRAWINGS

FIGS. 1A-1B depicts flow diagrams of the diagnosis of genetic diseases by standard and rapid genome sequencing. FIG. 1A is a flow diagram of the diagnosis of genetic diseases. FIG. 1B is a flow diagram of the diagnosis of genetic diseases.

FIGS. 2A-2B depicts diagrams showing clinical natural language processing can extract a more detailed phenome than manual electronic health record (EHR) review or Online Mendelian Inheritance in Man (OMIM) clinical synopsis. FIG. 2A is a schematic diagram. FIG. 2B is a schematic diagram.

FIGS. 3A-3H depicts a comparison of observed and expected phenotypic features of children with suspected genetic diseases. FIG. 3A is a graphical diagram depicting data. FIG. 3B is a graphical diagram depicting data. FIG. 3C is a graphical diagram depicting data. FIG. 3D is a Venn diagram depicting data. FIG. 3E is a graphical diagram depicting data. FIG. 3F is a graphical diagram depicting data. FIG. 3G is a graphical diagram depicting data. FIG. 3H is a Venn diagram depicting data.

FIG. 4 is a Venn diagram showing overlap of observed and expected patient phenotypic features in 95 children diagnosed with 97 genetic diseases.

FIGS. 5A-5B is a series of graphs depicting precision, recall, and F1-score of phenotypic features identified manually, by CNLP, and OMIM. FIG. 5A is a series of graphical diagrams depicting data. FIG. 5B is a series of graphical diagrams depicting data.

FIG. 6 is a flow diagram of the software components of the autonomous system for provisional diagnosis of genetic diseases by rapid genome sequencing in one embodiment of the invention.

### DETAILED DESCRIPTION OF THE INVENTION

The present invention is based on an innovative computational method and platform for genomic analysis. The invention provides a prototypic, autonomous system for rapid diagnosis of genetic diseases in intensive care unit populations. It performs clinical natural language processing (CNLP) to automatically identify deep phenomes of acutely ill children from electronic medical records (EMR). The method and platform described herein provides for clinical diagnosis of genetic diseases in a median of 20:10 hours that can be scaled to thirty patients per week per genome sequencing instrument, with automated provisional diagnosis of genetic diseases.

As discussed in detail in the Example, by informing timely targeted treatments, rapid genetic or genomic sequencing can improve the outcomes of seriously ill children with genetic diseases, particularly infants in neonatal and pediatric intensive care units (ICUs). The need for highly qualified professionals to decipher results, however, precludes widespread implementation.

In various embodiments, the present disclosure provides a platform for population-scale, provisional diagnosis of genetic diseases with automated phenotyping and interpretation. As detailed in the Example provided herein, genome sequencing was expedited by bead-based genome library preparation directly from blood, and sequencing of paired 100-nt reads in 15.5 hours. CNLP automatically extracted children's deep phenomes from electronic health records with 80% precision and 93% recall. In 101 children with 105 genetic diseases, a mean of 4.3 CNLP-extracted phenotypic features matched the expected phenotypic features of those diseases, compared with a match of 0.9 phenotypic features used in manual interpretation. Provisional diagnosis was automated by combining the ranking of the similarity of a patient's CNLP phenome with respect to the expected phenotypic features of all genetic diseases, together with the ranking of the pathogenicity of all of the patient's genomic variants. Automated, retrospective diagnoses concurred well with expert manual interpretation (97% recall, 99% precision in 95 children with 97 genetic diseases). Prospectively, the platform and method of the disclosure correctly diagnosed three of seven seriously ill ICU infants (100% precision and recall) with a mean time saving of 22:19 hours. In each case, the diagnosis impacted treatment. Genome sequencing with automated phenotyping and interpretation in a median 20:10 hours may increase adoption in ICUs, and, thereby, timely implementation of precise treatments.

Before the present compositions and methods are described, it is to be understood that this invention is not limited to particular methods and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Methods

In one aspect the invention provides a method for conducting genetic analysis. The analysis may be utilized to diagnose a disease or disorder, in particular a rare genetic disease. The method can also be utilized to rule out a genetic disease. The method of the invention is particularly useful in detecting and/or diagnosing a genetic disease in a subject that is less than 5 years old, such as an infant, neonate or fetus.

In embodiments the method includes:

a) determining a phenome of a subject from an electronic medical record (EMR), wherein the phenome includes a plurality of clinical phenotypes extracted from the EMR;

b) translating the clinical phenotypes into standardized vocabulary;

c) generating a first list of potential differential diagnoses of the subject;

d) performing genetic sequencing of a DNA sample from the subject;

e) determining genetic variants of the DNA;

f) analyzing the results of (c) and (e) to generate a second list of potential differential diagnoses of the subject, the second list being rank ordered; and

g) generating a report including results of the analysis of (f).

In embodiments, the method may further include generating the EMR for the subject prior to determining the phenome of the subject.

As used herein, “phenome” refers to the set of all phenotypes expressed by a cell, tissue, organ, organism, or species. The phenome represents an organisms' phenotypic traits.

As used herein, “EMR” refers to an electronic medical record and is used synonymously herein with “electronic health record” or “EHR”.

The method includes determining a phenome of a subject from an electronic medical record (EMR). This is performed by extracting a plurality of clinical phenotypes from the EMR. Natural language processing and/or automated feature extraction from non-standardized and standardized fields of the EMR of a subject is used to create a list of the clinical features of disease in that individual.

Translating the clinical phenotypes into standardized vocabulary is then performed utilizing a variety of computation methods known in the art. In one embodiment, translation is performed by natural language processing. This type of processing is utilized for translation and mining of non-structured text. Alternatively, data organized in discrete or structured fields may be retrieved/translated utilizing a conventional query language known in the art. Embodiments of standardized vocabularies include the Human Phenotype Ontology, Systematized Nomenclature of Medicine—Clinical Terms, and International Classification of Diseases—Clinical Modification.

The method also entails generating a first list of potential differential diagnoses of the subject. This is performed by query of a database populated with known clinical phenotypes expressed in the same vocabulary as the standardized vocabulary of the translated clinical phenotypes. Embodiments of databases of known clinical phenotypes include Online Mendelian Inheritance in Man—Clinical Synopsis, and Orphanet Clinical Signs and Symptoms. This list may be generated with an algorithm that rank orders all potential differential diagnoses based on goodness of fit. This list may also be generated with an algorithm that rank orders all potential differential diagnoses based on the sum of the distances of the observed and expected phenotypes in the standardized, hierarchical vocabulary.

Genetic variants are then determined from genomic sequencing performed on a DNA sample from the subject. In embodiments, this includes annotation and classification of the genetic variants. Annotation of all, or some, of the genetic variations in the subject's genome is performed to identify all variants that are of categories such as uncertain significance (VUS), pathogenic (P) or likely pathogenic (LP) and to retain genetic variations with an allele frequency of <5, 4, 3, 2, 1, 0.5, or 0.1% in a population of healthy individuals. The method may further include annotation of the genetic variants to identify and rank all diplotypes categorically, for example as being of uncertain significance (VUS), pathogenic (P) or likely pathogenic (LP) on the basis of pathogenicity. An embodiment of the classification system is the Joint Consensus Recommendation of the American College of Medical Genetics and Genomics and the Association for Molecular Pathology Standards and Guidelines for the Interpretation of Sequence Variants. The method may further include annotation of the pathogenicity of variants and diplotypes on a continuous, probabilistic scale, where a variant that is well established to be benign, for example, has a score of zero, and a variant that is well established to be pathogenic variant has a score of one, and likely benign, variants of uncertain significance, and likely pathogenic variants have scores between zero and one.

A second list of potential differential diagnoses of the subject is then generated by comparing the annotated VUS, LP and P diplotypes on a regional genomic basis with corresponding genomic regions associated with the first list of potential differential diagnoses. Genetic variants are ranked based on a combination of rank of goodness of fit of clinical phenotypes, rank of pathogenicity of diplotypes, and/or allele frequencies of the genetic variants in a population of health individuals. The list of potential differential diagnoses may further include annotation of their probability of being causative of the patient's condition on a continuous scale, rather than binary diagnosis/no diagnosis results.

In embodiments the genetic variants determined from the subject's genome may be utilized to generate a probabilistic diagnosis for use in generating the second list of potential diagnoses.

A report is then generated setting forth the potential differential diagnoses of the subject, preferably in order of score to identify the diagnosis with the highest probability.

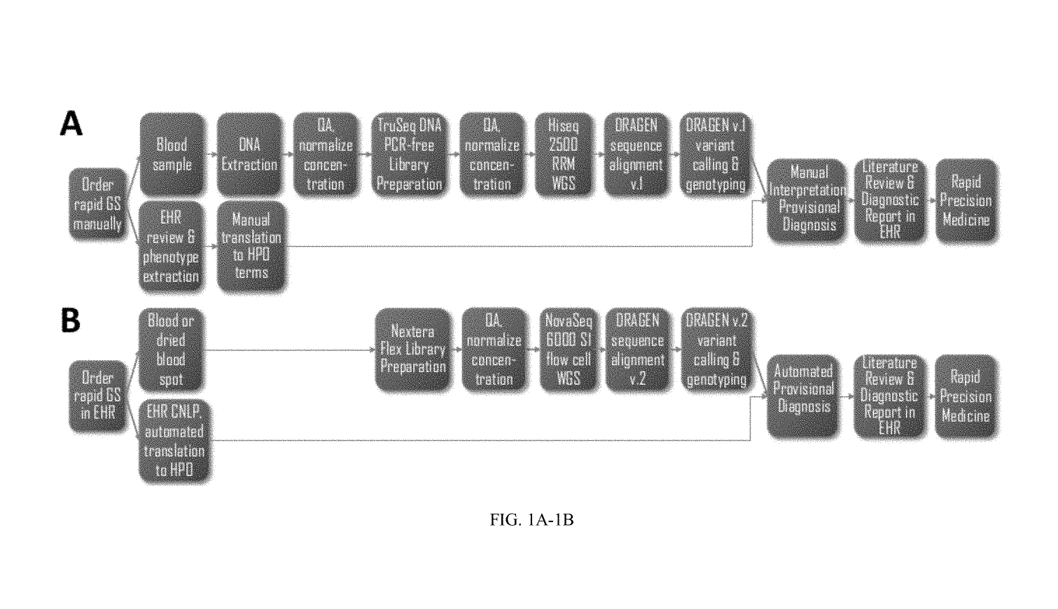

The method of the invention is illustrated in FIG. 1B. FIG. 1B is a flow chart showing AI involved automated extraction of the phenome from subject's EMR by clinical natural language processing (CNLP), translation from SNOMED-CT to Human Phenotype Ontology (HPO) terms (e.g., a standardized vocabulary), derivation of a comprehensive differential diagnosis gene list, identification of variants in genomic sequences, assembling those variants into likely pathogenic, causal diplotypes on a gene-by-gene basis, integration of the genotype and differential diagnosis lists, and retention of the highest ranking provisional diagnosis(es).

The method of present invention allows for a myriad of genetic analysis types to identify disease.

Methods described herein are useful in perinatal testing wherein the parental, e.g., maternal and/or paternal, genotypes are known. In an aspect, the methods are used to determine if a subject has inherited a deleterious combination of markers, e.g., mutations, from each parent putting the subject at risk for disease, e.g., Lesch-Nyhan syndrome. The disease may be an autosomal recessive disease, e.g., Spinal Muscular Atrophy. The disease may be X-linked, e.g., Fragile X syndrome. The disease may be a disease caused by a dominant mutation in a gene, e.g., Huntington's Disease. In some embodiments, the maternal nucleic acid sequence is the reference sequence. In some embodiments, the paternal nucleic acid sequence is the reference sequence. In some embodiments, the marker(s), e.g., mutation(s), are common to each parent. In some embodiments, the marker(s), e.g., mutation(s), are specific to one parent.

In some embodiments, haplotypes of an individual, such as maternal haplotypes, paternal haplotypes, or fetal haplotypes are constructed. The haplotypes comprise alleles co-located on the same chromosome of the individual. The process is also known as “haplotype phasing” or “phasing”. A haplotype may be any combination of one or more closely linked alleles inherited as a unit. The haplotypes may comprise different combinations of genetic variants. Artifacts as small as a single nucleotide polymorphism pair can delineate a distinct haplotype. Alternatively, the results from several loci could be referred to as a haplotype. For example, a haplotype can be a set of SNPs on a single chromatid that is statistically associated to be likely to be inherited as a unit.

In some embodiments, the maternal haplotype is used to distinguish between a fetal genetic variant and a maternal genetic variant, or to determine which of the two maternal chromosomal loci was inherited by the fetus.

In some embodiments, the methods provided herein may be used to detect the presence or absence of a genetic variant in a region of interest in the genome of a subject, such as an infant or fetus in a pregnant woman, wherein the fetal genetic variant is an X-linked recessive genetic variant. X-linked recessive disorders arise more frequently in male fetus because males with the disorder are hemizygous for the particular genetic variant. Example X-linked recessive disorders that can be detected using the methods described herein include Duchenne muscular dystrophy, Becker's muscular dystrophy, X-linked agammaglobulinemia, hemophilia A, and hemophilia B. These X-linked recessive variants can be inherited variants or de novo variants.

In some embodiments, provided herein is a method of detecting the presence or absence of a genetic variant in a region of interest in the genome of an infant or a fetus in a pregnant woman, wherein the fetal genetic variant is a de novo genetic variant or a paternally-inherited genetic variant. In some embodiments, the father's genome is sequenced to reveal whether the genetic variant is a paternally inherited genetic variant or a de novo genetic variant. That is, if the fetal genetic variant is not present in the father, and the described method indicates that the fetal genetic variant is distinguishable from the maternal genome, then the fetal genetic variant is a de novo variant. Accordingly, provided herein is a method of determining whether a fetal genetic variant is an inherited genetic variant or a de novo genetic variant. In some embodiments, the mother's genome is sequenced to reveal whether the genetic variant is a paternally inherited genetic variant or a de novo genetic variant. That is, if the fetal genetic variant is not present in the mother, and the described method indicates that the fetal genetic variant is distinguishable from the paternal genome, then the fetal genetic variant is a de novo variant. Accordingly, provided herein is a method of determining whether a fetal genetic variant is an inherited genetic variant or a de novo genetic variant.

In some embodiments, provided herein is a method of detecting the presence or absence of a genetic variant in a region of interest in the genome of an infant or a fetus in a pregnant woman, wherein the fetal genetic variant is a de novo copy number variant (such as a copy number loss variant) or a paternally-inherited copy number variant (such as a copy number loss variant). In some embodiments, the father's genome is sequenced to reveal whether the copy number variant is a paternally inherited copy number variant or a de novo copy number variant. That is, if the fetal copy number variant is not present in the father, and the described method indicates that the fetal copy number variant is distinguishable from the maternal genome, then the fetal copy number variant is a de novo copy number variant. Accordingly, provided herein is a method of determining whether a fetal copy number variant is an inherited copy number variant or a de novo copy number variant.

In some embodiments, the methods provided herein allow for detecting the presence or absence of a genetic variant in a region of interest in the genome of an infant or fetus in a pregnant woman, wherein the fetal genetic variant is an autosomal recessive fetal genetic variant. In some embodiments, the autosomal fetal genetic variant is an SNP. In some embodiments, the fetal genetic variant is a copy number variant, such as a copy number loss variant, or a microdeletion.

The method of the disclosure contemplates genetic sequencing. Sequencing may be by any method known in the art. Sequencing methods include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent™ sequencing), nanopore sequencing, pyrosequencing (454), sequencing by synthesis, sequencing by ligation (SOLiD™ sequencing), sequencing by electron microscopy, dideoxy sequencing reactions (Sanger method), massively parallel sequencing, polony sequencing, and DNA nanoball sequencing. In some embodiments, sequencing involves hybridizing a primer to the template to form a template/primer duplex, contacting the duplex with a polymerase enzyme in the presence of a detectably labeled nucleotides under conditions that permit the polymerase to add nucleotides to the primer in a template-dependent manner, detecting a signal from the incorporated labeled nucleotide, and sequentially repeating the contacting and detecting steps at least once, wherein sequential detection of incorporated labeled nucleotide determines the sequence of the nucleic acid. In some embodiments, the sequencing comprises obtaining paired end reads.

In some embodiments, sequencing of the nucleic acid from the sample is performed using whole genome sequencing (WGS) or rapid WGS (rWGS). In some embodiments, targeted sequencing is performed and may be either DNA or RNA sequencing. The targeted sequencing may be to a subset of the whole genome. In some embodiments the targeted sequencing is to introns, exons, non-coding sequences or a combination thereof. In other embodiments, targeted whole exome sequencing (WES) of the DNA from the sample is performed. The DNA is sequenced using a next generation sequencing platform (NGS), which is massively parallel sequencing. NGS technologies provide high throughput sequence information, and provide digital quantitative information, in that each sequence read that aligns to the sequence of interest is countable. In certain embodiments, clonally amplified DNA templates or single DNA molecules are sequenced in a massively parallel fashion within a flow cell (e.g., as described in WO 2014/015084). In addition to high-throughput sequence information, NGS provides quantitative information, in that each sequence read is countable and represents an individual clonal DNA template or a single DNA molecule. The sequencing technologies of NGS include pyrosequencing, sequencing-by-synthesis with reversible dye terminators, sequencing by oligonucleotide probe ligation and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. In embodiments, the methodology of the disclosure utilizes systems such as those provided by Illumina, Inc, (HiSeg™ X10, HiSeg™ 1000, HiSeq™ 2000, HiSeq™ 2500, HiSeq™ 4000, NovaSeq™ 6000, Genome Analyzers™, MiSeg™ systems), Applied Biosystems Life Technologies (ABI PRISM™ Sequence detection systems, SOLiD™ System, Ion PGM™ Sequencer, ion Proton™ Sequencer).

In some embodiments, rWGS of DNA is performed. In some embodiments, rWGS is performed on samples of the subject, e.g., an infant, neonate or fetus. In some embodiments, rWGS is performed on maternal samples along with that of the subject. In some embodiments, rWGS is performed on paternal samples along with that of the subject. In some embodiments, rWGS is performed on maternal and paternal samples along with that of the subject.

In some embodiments, rapid whole exome sequencing (rWES) of DNA is performed. In some embodiments, rWES is performed on samples of the subject, e.g., an infant, neonate or fetus. In some embodiments, rWES is performed on maternal samples along with that of the subject. In some embodiments, rWES is performed on paternal samples along with that of the subject. In some embodiments, rWES is performed on maternal and paternal samples along with that of the subject.

As used herein, the term “mutation” herein refers to a change introduced into a reference sequence, including, but not limited to, substitutions, insertions, deletions (including truncations) relative to the reference sequence. Mutations can involve large sections of DNA (e.g., copy number variation). Mutations can involve whole chromosomes (e.g., aneuploidy). Mutations can involve small sections of DNA. Examples of mutations involving small sections of DNA include, e.g., point mutations or single nucleotide polymorphisms (SNPs), multiple nucleotide polymorphisms, insertions (e.g., insertion of one or more nucleotides at a locus but less than the entire locus), multiple nucleotide changes, deletions (e.g., deletion of one or more nucleotides at a locus), and inversions (e.g., reversal of a sequence of one or more nucleotides). The consequences of a mutation include, but are not limited to, the creation of a new character, property, function, phenotype or trait not found in the protein encoded by the reference sequence. In some embodiments, the reference sequence is a parental sequence. In some embodiments, the reference sequence is a reference human genome, e.g., h19. In some embodiments, the reference sequence is derived from a non-cancer (or non-tumor) sequence. In some embodiments, the mutation is inherited. In some embodiments, the mutation is spontaneous or de novo.

As used herein, a “gene” refers to a DNA segment that is involved in producing a polypeptide and includes regions preceding and following the coding regions as well as intervening sequences (introns) between individual coding segments (exons).

The terms “polynucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. Polynucleotides may be single- or multi-stranded (e.g., single-stranded, double-stranded, and triple-helical) and contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, and the like). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need and circular portions. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may include modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component, tag, reactive moiety, or binding partner. Polynucleotide sequences, when provided, are listed in the 5′ to 3′ direction, unless stated otherwise.

As used herein, “polypeptide” refers to a composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may include modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, synthetic amino acids and the like), as well as other modifications known in the art.

As used herein, the term “sample” herein refers to any substance containing or presumed to contain nucleic acid. The sample can be a biological sample obtained from a subject. The nucleic acids can be RNA, DNA, e.g., genomic DNA, mitochondrial DNA, viral DNA, synthetic DNA, or cDNA reverse transcribed from RNA. The nucleic acids in a nucleic acid sample generally serve as templates for extension of a hybridized primer. In some embodiments, the biological sample is a biological fluid sample. The fluid sample can be whole blood, plasma, serum, ascites, cerebrospinal fluid, sweat, urine, tears, saliva, buccal sample, cavity rinse, feces or organ rinse. The fluid sample can be an essentially cell-free liquid sample (e.g., plasma, serum, sweat, urine, and tears). In other embodiments, the biological sample is a solid biological sample, e.g., feces or tissue biopsy, e.g., a tumor biopsy. A sample can also comprise in vitro cell culture constituents (including but not limited to conditioned medium resulting from the growth of cells in cell culture medium, recombinant cells and cell components). In some embodiments, the sample is a biological sample that is a mixture of nucleic acids from multiple sources, i.e., there is more than one contributor to a biological sample, e.g., two or more individuals. In one embodiment the biological sample is a dried blood spot.

In the present invention, the subject is typically a human but also can be any species with methylation marks on its genome, including, but not limited to, a dog, cat, rabbit, cow, bird, rat, horse, pig, or monkey. In one embodiment, the subject is a human child. In some embodiments, the child is less than 5, 4, 3, 2 or 1 year of age. In embodiments, the subject is an infant, neonate or fetus.

Computer Systems

The present invention is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions. In addition, although the invention is described in the medical diagnosis context, the present invention may be practiced in conjunction with any number of applications, environments and data analyses; the systems described herein are merely exemplary applications for the invention.

Methods for genetic analysis according to various aspects of the present invention may be implemented in any suitable manner, for example using a computer program operating on the computer system. An exemplary genetic analysis system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, comprise any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing genetic information may be implemented in a single device or implemented in a plurality of devices. The software may be accessible via a network such that storage and processing of information takes place remotely with respect to users. The genetic analysis system according to various aspects of the present invention and its various elements provide functions and operations to facilitate genetic analysis, such as data gathering, processing, analysis, reporting and/or diagnosis. The present genetic analysis system maintains information relating to samples and facilitates analysis and/or diagnosis, For example, in the present embodiment, the computer system executes the computer program, which may receive, store, search, analyze, and report information relating to the genome. The computer program may comprise multiple modules performing various functions or operations, such as a processing module for processing raw data and generating supplemental data and an analysis module for analyzing raw data and supplemental data to generate a disease status model and/or diagnosis information.

The procedures performed by the genetic analysis system may comprise any suitable processes to facilitate genetic analysis and/or disease diagnosis. In one embodiment, the genetic analysis system is configured to establish a disease status model and/or determine disease status in a patient. Determining or identifying disease status may comprise generating any useful information regarding the condition of the patient relative to the disease, such as performing a diagnosis, providing information helpful to a diagnosis, assessing the stage or progress of a disease, identifying a condition that may indicate a susceptibility to the disease, identify whether further tests may be recommended, predicting and/or assessing the efficacy of one or more treatment programs, or otherwise assessing the disease status, likelihood of disease, or other health aspect of the patient.

The genetic analysis system may also provide various additional modules and/or individual functions. For example, the genetic analysis system may also include a reporting function, for example to provide information relating to the processing and analysis functions. The genetic analysis system may also provide various administrative and management functions, such as controlling access and performing other administrative functions. The genetic analysis system may also provide clinical decision support, to assist the physician in the provision of individualized genomic or precision medicine for the analyzed patient.

The genetic analysis system suitably generates a disease status model and/or provides a diagnosis for a patient based on genomic data and/or additional subject data relating to the subject's health or well-being. The genetic data may be acquired from any suitable biological samples.

The following example is provided to further illustrate the advantages and features of the present invention, but it is not intended to limit the scope of the invention. While this example is typical of those that might be used, other procedures, methodologies, or techniques known to those skilled in the art may alternatively be used.

### EXAMPLES

### Example I

### Rapid Genome Sequencing for Genetic Disease Diagnosis

In this example, a prototypic, autonomous system for rapid diagnosis of genetic diseases in intensive care unit populations is described. It performs clinical natural language processing (CNLP) to automatically identify deep phenomes of acutely ill children from electronic medical records (EMR).

Experimental Materials and Methods

Study Design.

This study was designed to furnish training and test datasets to assist in the development of a prototypic, autonomous system for very rapid, population-scale, provisional diagnoses of genetic diseases by genomic sequencing, and separate datasets to test the analytic and diagnostic performance of the resultant system both retrospectively and prospectively. The 401 subjects analyzed herein were a convenience sample of the first symptomatic children who were enrolled in four studies that examined the diagnostic rate, time to diagnosis, clinical utility of diagnosis, outcomes, and healthcare utilization of rapid genomic sequencing at Rady Children's Hospital, San Diego, USA (ClinicalTrials.gov Identifiers: NCT03211039, NCT02917460, and NCT03385876) (18, 22-24, 28, 30). One of the studies was a randomized controlled trial of genome and exome sequencing (NCT03211039); the others were cohort studies. All subjects had a symptomatic illness of unknown etiology in which a genetic disorder was suspected. All subjects had a Rady Children's Hospital Epic EHR and a genomic sequence (genome or exome) that had been interpreted manually for diagnosis of a genetic disease. They included five groups, namely, 16 children tested for genetic diseases by rapid whole genome sequencing whose EHRs were used to train CNLP (Table 4), ten children with genetic diseases diagnosed by rapid genomic sequencing whose EHRs were used to test the performance of CNLP (Table 5), 101 children with genetic diseases diagnosed by rapid genomic sequencing whose genomic sequences and EHRs were used to test the retrospective performance of the autonomous diagnostic system, seven seriously ill children with suspected genetic diseases whose DNA samples and EHRs were used to test the prospective performance of the autonomous diagnostic system (Table 1), and 274 control children in whom rapid genomic sequencing did not disclose a genetic disease diagnosis.

Standard, Clinical, Rapid Whole Genome and Exome Sequencing, Analysis and Interpretation.

Standard, clinical, rWGS and rWES were performed in laboratories accredited by the College of American Pathologists (CAP) and certified through Clinical Laboratory Improvement Amendments (CLIA). Experts selected key clinical features representative of each child's illness from the Epic EHR and mapped them to genetic diagnoses with Phenomizer™ or Phenolyzer™ (16, 18, 20-24, 45, 63). Trio EDTA-blood samples were obtained where possible. Genomic DNA was isolated with an EZ1 Advanced XL™ robot and the EZ1 DSP DNA™ Blood kit (Qiagen). DNA quality was assessed with the Quant-iT Picogreen dsDNA™ assay kit (ThermoFisher Scientific) using the Gemini EM Microplate Reader™ (Molecular Devices). Genomic DNA was fragmented by sonication (Covaris) and bar-coded, paired-end, PCR-free libraries were prepared for rWGS with TruSeq DNA LT™ kits (Illumina) or Hyper kits (KAPA Biosystems). Sequencing libraries were analyzed with a Library Quantification Kit™ (KAPA Biosystems) and High Sensitivity NGS Fragment Analysis Kit™ (Advanced Analytical), respectively. Paired-end 101 nt rWGS was performed to 45-fold coverage with Illumina HiSeg™ 2500 (rapid run mode), HiSeg™ 4000, or NovaSeg™ 6000 (S2 flow cell) instruments, as described (16). rWES was performed by GeneDx™. Exome enrichment was with the xGen Exome Research Panel™ v1.0 (Integrated DNA Technologies), and amplification used the Herculase II Fusion™ polymerase (Agilent) (18, 64). Sequences were aligned to human genome assembly GRCh37 (hg19), and variants were identified with the DRAGEN™ Platform (v.2.5.1, Illumina, San Diego) (16). Structural variants were identified with Manta™ and CNVnator™ (using DNAnexus™), a combination that provided the highest sensitivity and precision in 21 samples with known structural variants (Table 6) (18, 65, 66). Structural variants were filtered to retain those affecting coding regions of known disease genes and with allele frequencies <2% in the RCIGM database. Nucleotide and structural variants were annotated, analyzed, and interpreted by clinical molecular geneticists using Opal Clinical™ (Fabric Genomics), according to standard guidelines (50, 67). Opal™ annotated variants with respect to pathogenicity, generated a rank ordered differential diagnosis based on the disease gene algorithm VAAST, a gene burden test, and the algorithm PHEVOR (Phenotype Driven Variant Ontological Re-ranking), which combined the observed HPO phenotype terms from patients, and re-ranked disease genes based on the phenotypic match and the gene score (68-70). Automatically generated, ranked results were manual interpreted through iterative Opal searches. Initially, variants were filtered to retain those with allele frequencies of <1% in the Exome Variant Server™, 1000 Genomes Samples™, and Exome Aggregation Consortium™ database (71). Variants were further filtered for de novo, recessive and dominant inheritance patterns. The evidence supporting a diagnosis was then manually evaluated by comparison with the published literature. Analysis, interpretation and reporting required an average of six hours of expert effort. If rWGS or rWES established a provisional diagnosis for which a specific treatment was available to prevent morbidity or mortality, this was immediately conveyed to the clinical team, as described. All causative variants were confirmed by Sanger sequencing or chromosomal microarray, as appropriate. Secondary findings were not reported, but medically actionable incidental findings were reported if families consented to receiving this information.

Natural Language Processing and Phenotype Extraction.

Extraction of HPO terms from the EHR entailed four steps as follows.

1) Clinical records were exported from the EHR data warehouse, transformed into a compatible format (JSON) and loaded into CLiX ENRICH™.

2) A semi-automated query map was created, using HPO terms (and their synonyms) as the input and CLiX queries as the output. The HPO terms were passed through the CLiX encoding engine, resulting in creation of CLiX post-coordinated SNOMED™ expressions for each recognized HPO term or synonym. Where matches were not exact, manual review was used to validate the generated CLiX™ queries. Where there was no match or incorrect matches, new content was added to the Clinithink SNOMED™ extension and terminology files to ensure appropriate matches between phenotypes in HPO and those in SNOMED-CT™. This was an iterative process that resulted in a CLiX™ query set that covered 60% (7,706) of 12,786 HPO terms (Oct. 9, 2017 HPO build).

3) EHR documents containing unstructured data were passed through the CNLP engine. The natural language processing engine read the unstructured text and encoded it in structured format as post-coordinated SNOMED expressions as shown in the example below which corresponds to HP0007973, retinal dysplasia:

243796009|Situation with explicit context|: {408731000|Temporal
context|=410511007|Current or past|, 246090004|Associated finding|=95494009|Retinal
dysplasia|, 408732007|Subject relationship context|=410604004|Subject of record|,
408729009|Finding context|=410515003|Known present|}

Each SNOMED expression is made up of several parts, including the associated clinical finding, the temporal context, finding context and subject context all contained within the situational wrapper. Capturing fully post-coordinated SNOMED expressions ensures that the correct context of the clinical note is preserved. Some HPO phenotypes cannot be found in SNOMED and can only be represented using post-coordinated expressions, as shown in the following example which is the encoding of HP0008020, progressive cone dystrophy:

243796009|Situation with explicit context|: {408731000|Temporal
context|=410511007|Current or past|, 246090004|Associated finding|=(312917007|Cone
dystrophy|:263502005|Clinical course|=255314001|Progressive|), 408732007|Subject
relationship context|=410604004|Subject of record|, 408729009|Finding
context|=410515003|Known present|}

Here, an additional attribute for ‘Clinical Course’ and an appropriate value, ‘Progressive’, are used to further qualify the expression. Clinithink™ used references to these SNOMED™ expressions, linked with Boolean logic, to create the queries corresponding to HPO terms. Shown below is an example query for HP0008866, failure to thrive secondary to recurrent infections:

```
    c*hp0008866_Failure_to_thrive_secondary_to_recurrent_infections
(hp0008866_1_1_Failure_to_thrive_q AND hp0002719_1_1_Infection_Recurrent_q)
q-hp0008866_1_1_Failure_to_thrive_q 243796009|Situation with explicit
context|:{408731000|Temporal context|=410511007|Current or past|,246090004|Associated
finding|=54840006|Failure to thrive|,408732007|Subject relationship
context|=410604004|Subject of record|,408729009|Finding context|=410515003|Known
present|}
    q-hp0002719_1_1_Infection_Recurrent_q 243796009|Situation with explicit
context|:{408731000|Temporal context|=410511007|Current or past|,246090004|Associated
finding|=(40733004|Infection|:263502005|Clinical
course|=255227004|Recurrent|),408732007|Subject relationship context|=410604004|Subject
of record|,408729009|Finding context|=410515003|Known present|}
```

For an encoding created from the unstructured data to trigger one of these queries, all of the components must be matched. Therefore, the encoding of a clinical note describing an affected sibling will not trigger the query since the encoding is that of family history whilst the query looks for the term in the subject of the record (i.e. the patient). Furthermore, it should be noted that some individual HPO synonyms generate more than one SNOMED™ expression. Therefore, each query used in the query set is a compound of often more than 2 SNOMED™ expressions. If the above constants are stripped out from each expression (the associated clinical finding, the temporal context, finding context and subject context all contained within the situational wrapper) from each expression in the query set (along with all of the associated SNOMED™ codes), the inventors can create a more readable format to show linguistically what is included in each query created by Clinithink™.

4) This encoded data was then interrogated by the CLiX™ query technology (abstraction). To trigger an HPO query, the encoded data had to either contain an exact match, or one of its logical descendants (exploiting the parent child hierarchy of the SNOMED™ ontology), resulting in a list of HPO terms for each patient.

rWGS.

Sequencing libraries were prepared from 10 μL of EDTA blood or five 3-mm punches from a Nucleic-Card Matrix™ dried blood spot (ThermoFisher) with Nextera DNA Flex Library Prep™ kits (Illumina) and five cycles of PCR, as described (35). For structural variant analysis, libraries were prepared by Hyper™ kits (KAPA Biosystems), as described above. Libraries were quantified with Quant-iT Picogreen dsDNA™ assays (ThermoFisher). Libraries were sequenced (2×101 nt) without indexing on the 51 FC with Novaseg™ 6000 S1 reagent kits (Illumina). Sequences were aligned to human genome assembly GRCh37 (hg19), and nucleotide variants were identified with the DRAGEN™ Platform (v.2.5.1, Illumina) (16).

Automated Tertiary Analysis.

Automated variant interpretation was performed using MOON™ (Diploid) (72). Data sources and versions were ClinVar: 2018-04-29; dbNSFP: 3.5; dbSNP: 150; dbscSNV: 1.1; Apollo: 2018-07-20; Ensembl: 37; gnomAD: 2.0.1; HPO: 2017-10-05; DGV: 2016-03-01; dbVar: 2018-06-24; MOON: 2.0.5). MOON™ generated a list of potential provisional diagnoses by sequentially filtering and ranking variants using decision trees, Bayesian models, neural networks, and natural language processing. MOON™ was iteratively trained with thousands of prior patient samples uploaded by prior investigators. No samples analysed in this study were used in training of MOON™.

The filtering pipeline was designed to minimize false negatives. For SNV analysis, MOON™ excluded low quality and common variants (>2% in gnomAD), and known Likely benign/Benign variants in ClinVar™. Only variants in coding regions, splice site regions and known pathogenic variants in non-coding regions were retained. A disease annotation was added to the remaining variants based on a proprietary disorder model (72). The disorder model performs natural language processing of the genetics literature to automatically extract associations between diseases, disease genes, inheritance patterns, specific clinical features, and other metadata on an ongoing basis.

Subsequent steps included filtering on variant frequency, with variable frequency thresholds depending on the inheritance pattern of the associated disease, known pathogenicity of the variant, and typical age of onset range of the annotated disease. In family analyses (duo/trio analysis), co-segregation of the variant with the phenotype, according to autosomal dominant, autosomal recessive, X-linked dominant or X-linked recessive inheritance patterns, was taken into account. Parent-child variant segregation was not applied as a strict filter criterion, thereby also ensuring that causal mutations following non-Mendelian inheritance (eg. with incomplete penetrance) were identified in family analyses. For proband-only analyses, only variants for which the zygosity of the called variant fit the inheritance pattern of the annotated disease were retained. In a final filter step, the phenotype overlap was scored between the input HPO terms describing the patient's phenotype and known disease manifestations of the annotated disorder annotated from the published literature. Variants in genes for which the phenotype match with the annotated disease was considered too limited based on Apollo™ were removed from the analysis. The final rank of variants was based on proprietary algorithms that took phenotype match and variant effect into account. In addition, MOON™ provided all metadata supporting the pathogenicity of ranked variants. MOON™ also returned an annotated list of all rare variants (<2% in gnomAD) and carrier status for recessive disorders.

For structural variant analysis, MOON™ removed known benign SV based on the Database of Genomic Variants™ (DGV). SVs overlapping pathogenic SVs listed in dbVar were retained for analysis. From the remaining variants, MOON™ discarded SV that did not overlap with coding regions of known disease genes (Apollo™). If a family analysis was performed, segregation of the SV was taken into account, although non-Mendelian inheritance patterns (for example, incomplete penetrance) were also supported. In a final filter step, only SVs for which there was phenotype overlap between the input HPO terms and known disease presentations of at least one of the genes affected by the SV, were retained. MOON™ then reported a ranked list of candidate SV, where ranking was mostly based on phenotype overlap.

Statistical Analysis.

To assess the complexity of phenomes associated with childhood genetic diseases, the inventors compared phenotypes identified by manual review, CNLP, and listed for each patient's diagnosis in OMIM. All analyses were conducted in R v3.3.3 (73). When applying CNLP to a patient's EHR, the list of HPO terms produced contained both terms that had an exact match to a phenotype in the clinical notes and terms that were superclasses (ancestor terms) of exact matches. The R package Ontologylndex™ v2.4 was used to load the October 2017 build of HPO into R and calculate the IC of each HPO term in the entire OMIM corpus (74). The IC for term phenotype, which reflects its clinical specificity, is given by IC(phenotype)=−log (p_{phenotype}), where p_{phenotype} was the probability of observing the exact term or one of its subclasses across all diseases in OMIM™. Since phenotypes that were extracted manually and by CNLP were restricted to subclasses of ‘Phenotypic abnormality’ (HP:0000118), OMIM™ terms that were subclasses of ‘Clinical Modifier’ (HP:0012823), ‘Frequency’ (HP:0040279), ‘Mode of inheritance’ (HP:0000005), and ‘Mortality/Aging’ (HP:0040006) were not included in the analyses. Phenotype sets were first compared visually by plotting the HPO graph for each patient with the R package hpoPlot™ v2.4 (75). Summary statistics for outcomes of interest include the mean, standard deviation (SD), and range. Prior to testing for significant differences, outcome variables were tested for normality using the Shapiro-Wilk test. Due to deviations from normality, differences in phenotype counts and IC were evaluated with 2-sided Mann-Whitney U tests and when the data were paired, Wilcoxon signed-rank tests. Correlation was assessed with Spearman's rank correlation coefficient (rₛ). Precision and recall were given by tp/(tp+fp) and tp/(tp+fn), respectively, where tp were true positives, fp were false positives, and fn were false negatives. The number of true positives, tp, was defined in two ways. First, tp was set to the number of HPO terms that overlapped between sets of phenotypes. Second, tp was calculated based on terms that were up to one degree of separation apart within the HPO hierarchy (parent-child terms) between sets of phenotypes, allowing for inexact, but similar, matches. Additional graphics were produced with packages ggplot2 v 2.2.1 and eulerr v4.0.0 (76, 77). A significance cutoff of p<0.05 was used for all analyses.

Results

Rapid Genome Sequencing for Genetic Disease Diagnosis.

In light of the limitations of current methods of rapid genomic sequencing, the inventors developed an automated platform for rapid, high throughput, provisional diagnosis of genetic diseases with genome sequencing by automating and accelerating our conventional workflow (FIG. 1). Conventional clinical genome sequencing requires preparatory steps of manual purification of genomic DNA from blood, DNA quality assessment, normalization of DNA concentration, sequencing library preparation, and library quality assessment (FIG. 1A). Instead, the inventors manually prepared sequencing libraries directly from blood or dried blood spots using microbeads to which transposons were attached (Nextera DNA Flex Library Prep Kit™, Illumina, Inc.; FIG. 1B) (35), as this method was both faster and less labor intensive. Of note, dried blood spots are the sample type used in mandatory newborn screening worldwide. In four timed runs with retrospective samples, manual Nextera™ library preparation from dried blood spots took a mean of 2 hours and 45 minutes, compared with at least 10 hours by conventional DNA purification and library preparation (Truseq DNA PCR-free Library Prep Kit™, Illumina, Inc.; Table 1). As with standard methods, Nextera Flex™ allowed samples to be prepared in batches and was amenable to automation with liquid-handling robots.

Following the preparatory steps, our previous method performed rapid genome sequencing with the HiSeg™ 2500 sequencer (Illumina) in rapid run mode, with one sample sequenced per sequencing instrument (˜120 gigabases (Gb) of 2×101 nt) in ˜25 hours (FIG. 1A) (16, 17). Here the inventors instead performed rapid genome sequencing with the NovaSeg™ 6000 sequencer and S1 flow cell (Illumina) (FIG. 1B), as this instrument was faster and less labor-intensive, requiring fewer steps to set up a sequencing run and automatically washing the instrument after a run. In four timed runs with retrospective samples, 2×101 nt genome sequencing took a mean 15:32 hours and yielded 404-537 Gb per flow cell, sufficient for 2-3 40× genome sequences (Table 1, Ttable 2).

Dynamic Read Analysis for GENomics™ (DRAGEN™, Illumina) is a hardware and software platform for alignment and variant calling that has been highly optimized for speed, sensitivity and accuracy (16). The inventors wrote scripts to automate the transfer of files from the sequencer to the DRAGEN™ platform. The DRAGEN™ platform then automatically aligned the reads to the reference genome and identified and genotyped nucleotide variants. Alignment and variant calling took a median of 1 hour for 150 Gb of paired-end 101 nt sequences (primary and secondary analysis, Table 1). Analytic performance of this new method, from blood sample receipt to output of genomic variant genotypes, was similar to standard clinical methods with reference human genome samples, retrospective patient samples, and prospective patient samples, except for lower sensitivity in the detection of nucleotide insertions/deletions (Table 2, Table 3). The new method did not assess structural variations.

CNLP of Electronic Health Records (EHRs).

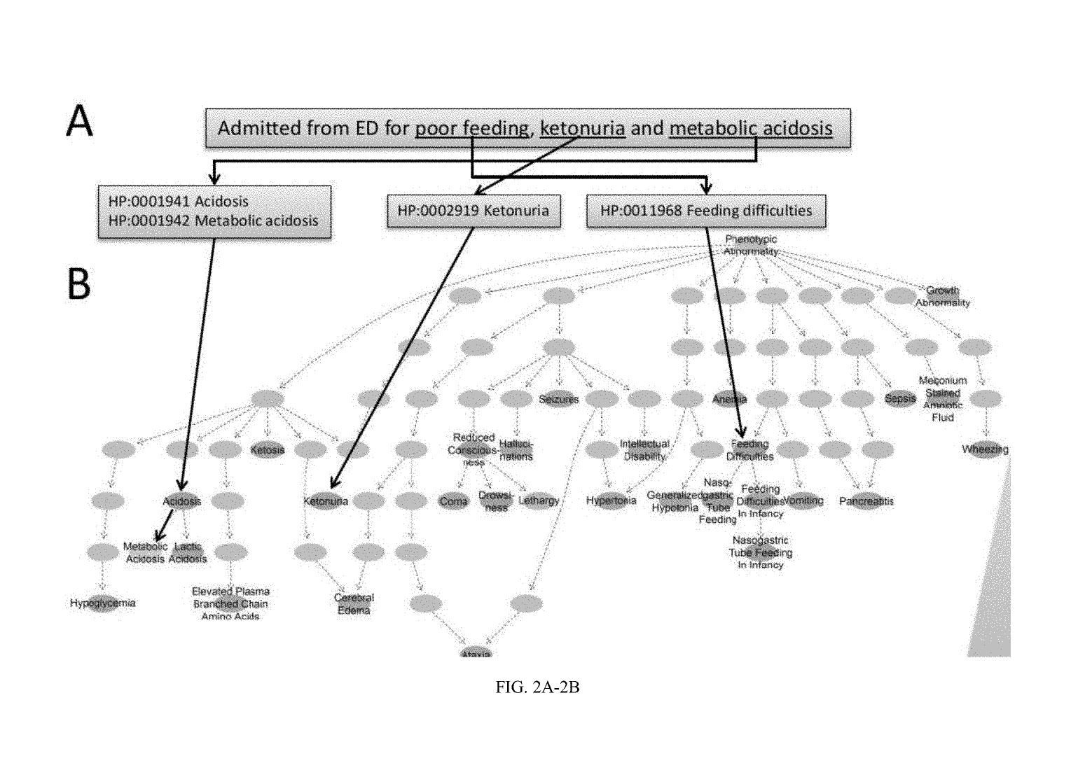

Genetic disease diagnosis requires determination of a differential diagnosis based on the overlap of the observed clinical features of a child's illness (phenotypic features) with the expected features of all genetic diseases. However, comprehensive EHR review can take hours. Additionally, manual phenotypic feature selection can be sparse and subjective (36, 37), and even expert reviewers can carry an unwritten bias into interpretation (FIG. 1A). The inventors sought automated, complete phenotypic feature extraction from EHRs, unbiased by expert opinion. The simplest approach would be to extract universal, structured phenotypic features, such as International Classification of Diseases (ICD) medical diagnosis codes, or Diagnosis Related Group (DRG) codes. However, these are sparse and lack sufficient specificity (38, 39). Instead, the inventors extracted clinical features from unstructured text in patient EHRs by CNLP that the inventors optimized for identification of patients with orphan diseases (CLiX ENRICH™, Clinithink Ltd.) (FIG. 1B, 2A). The inventors then iteratively optimized the protocol for the Rady Children's Hospital Epic EHRs using a training set of sixteen children who had received genomic sequencing for genetic disease diagnosis (Table 4). The standard output from CLiX ENRICH™ is in the form of Systematized Nomenclature of Medicine Clinical Terms (SNOMED-CT™). However, our automated methods required phenotypic features described in the Human Phenotype Ontology (HPO), a hierarchical reference vocabulary designed for description of the clinical features of genetic diseases (FIG. 2B). For this reason, the inventors mapped 7,706 (60%) of 12,786 HPO terms (13,685 including synonyms) and 75.4% of Orphanet Rare Disease HPO terms (June 2018 release) to SNOMED-CT™ by lexical and logical methods and then manually verified them. This enabled automated translation of phenotypic features extracted from the EHR by CNLP from SNOMED-CT concepts to HPO terms (FIG. 1B). In contrast, Dhombres et al. (2016) mapped 92% of HPO terms to SNOMED-CT, but only 49% were shown to be ontologically valid and clinically relevant (40).

The performance of the optimized CNLP was tested with the EHRs of ten test children who had received genomic sequencing for genetic disease diagnosis. The training and test sets did not overlap. Both exact EHR phenotypic feature matches and their hierarchical root terms were extracted from first record until time of enrollment for genomic sequencing. CNLP identified a mean of 86.7 phenotypic features (standard deviation (SD) 32.8, range 26-158; Table 5) in approximately 20 seconds per patient. A detailed manual review of the EHR was performed to identify all true positive, false positive and false negative CNLP phenotypic features in the test children. Based on this, the precision (positive predictive value, PPV) of CNLP was 0.80 (SD 0.13, range 0.50-0.93) and recall (sensitivity) was 0.93 (SD 0.02, range 0.91-0.96; Table 5), which were superior to prior CNLP-based extraction of HPO terms (36, 41). The principal reasons for false positives (FP) were: 1) incorrect CLiX™ encoding (n=89, 38% of 237 phenotypic features) due to misinterpreted context (n=31), unrecognized headings (n=23), incorrect acronym expansion (n=21), incorrect interpretation of a clinical word (n=8), or incorrectly attributed finding site for disease (n=6); 2) ambiguity of source text (unrecognized or incorrect syntax, abbreviations, acronyms or terminology; n=46, 19% of 237); 3) incongruity between SNOMED/HPO/clinical acumen (n=20, 8%); 4) failure to recognize a pasted citation as non-clinical text (n=68, 29%); and, 5) incorrect query logic (n=14, 6%) (Table 5).

Characterization of the CNLP-Derived Phenomes of Children with Suspected Genetic Diseases.

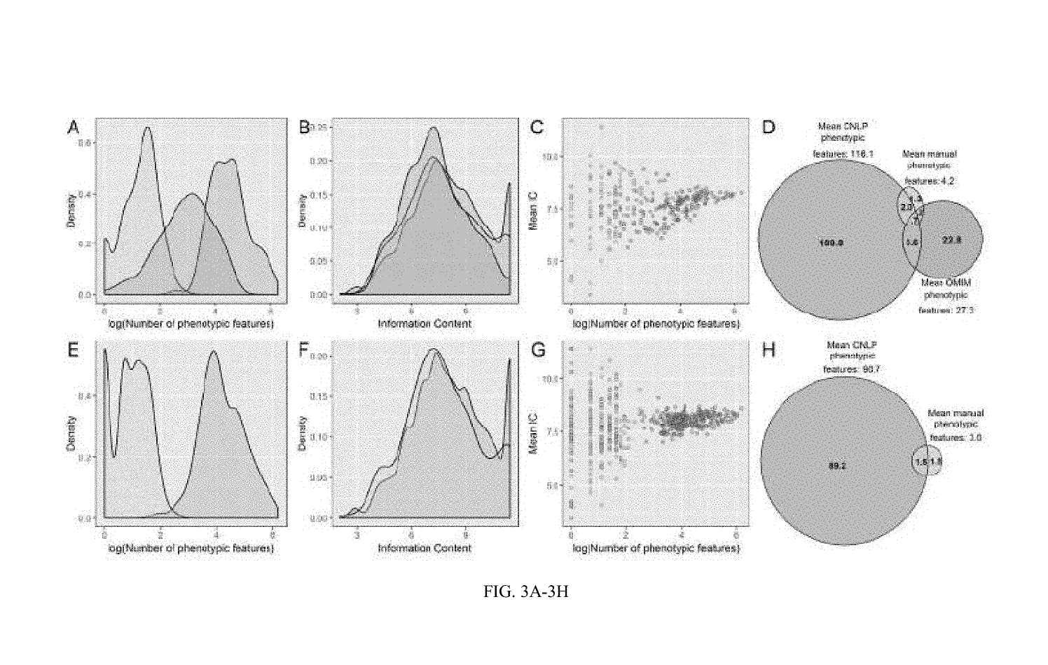

Development of an autonomous diagnostic system has been hindered by a dearth of knowledge of the topography of the phenomes of children with suspected genetic diseases (36, 42-44). Therefore the inventors compared EHR CNLP-derived phenomes with the comparatively sparse phenotypic features selected by experts during manual interpretation of the first 375 symptomatic children to receive genomic sequencing for diagnosis of genetic diseases at Rady Children's Hospital (101 children diagnosed with genomic sequencing: FIG. 3. A-D, 274 children that were not diagnosed: FIG. 3E-H). In 101 of these children, who had received genomic diagnoses of 105 genetic diseases (four had dual diagnoses), the inventors also compared the observed phenotypic features with the expected phenotypic features for those diseases, obtained from the Clinical Synopsis field of Online Mendelian Inheritance in Man (OMIM) (18, 22-24, 41). In the 101 diagnosed children, CNLP identified 27-fold more phenotypic features (mean 116.1, SD 93.6, range 13-521) than expert manual selection at interpretation (mean 4.2, SD 2.6, range 1-16), and 4-fold more than OMIM (mean 27.3, SD 22.8, range 1-100; FIG. 3A, 3D) (45, 46) Similarly, prior studies demonstrated 2-fold more phenotypic features extracted by CNLP than comprehensive, expert manual extraction (36), and 18-fold more phenotypic features extracted by CNLP than Orphanet HPO terms for those diseases (47). CNLP extracted more phenotypic features in the 101 diagnosed children than the 274 undiagnosed children (mean, 116.1 vs 90.7, respectively; P=0.0004, Mann-Whitney U test; FIG. 3A, 3D, 3E, 3H). This suggested the possibility that undiagnosed children, in part, did not have enough detail in their medical records to make a molecular diagnosis. In addition, there was greater overlap between CNLP- and manually-extracted phenotypic features in diagnosed children (mean 2.74 terms, SD 1.7, range 0-9) than undiagnosed (mean 1.52 terms, SD 1.48, range 0-7; P<0.0001, Mann-Whitney U test; FIG. 3D, 3H). This suggested that undiagnosed children, in part, had less consistent information on phenotypic features.

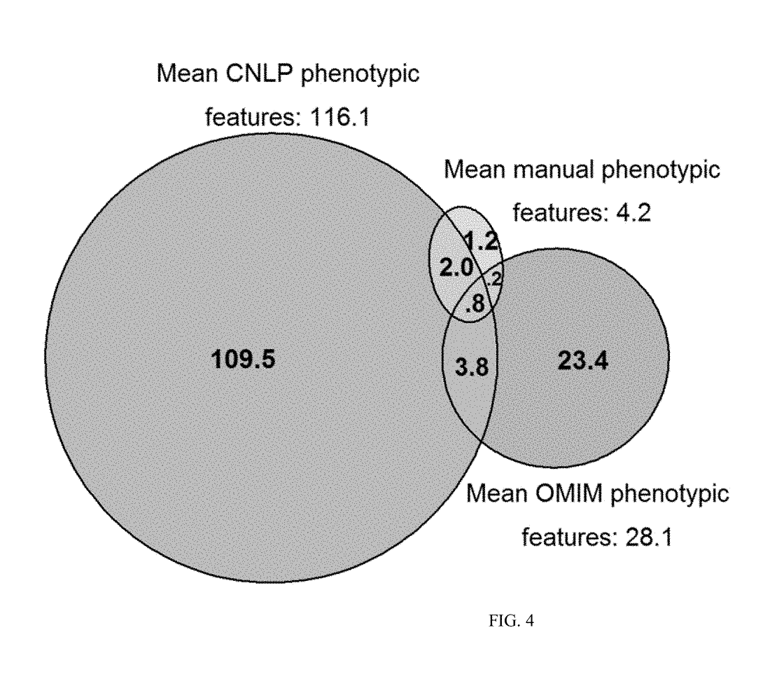

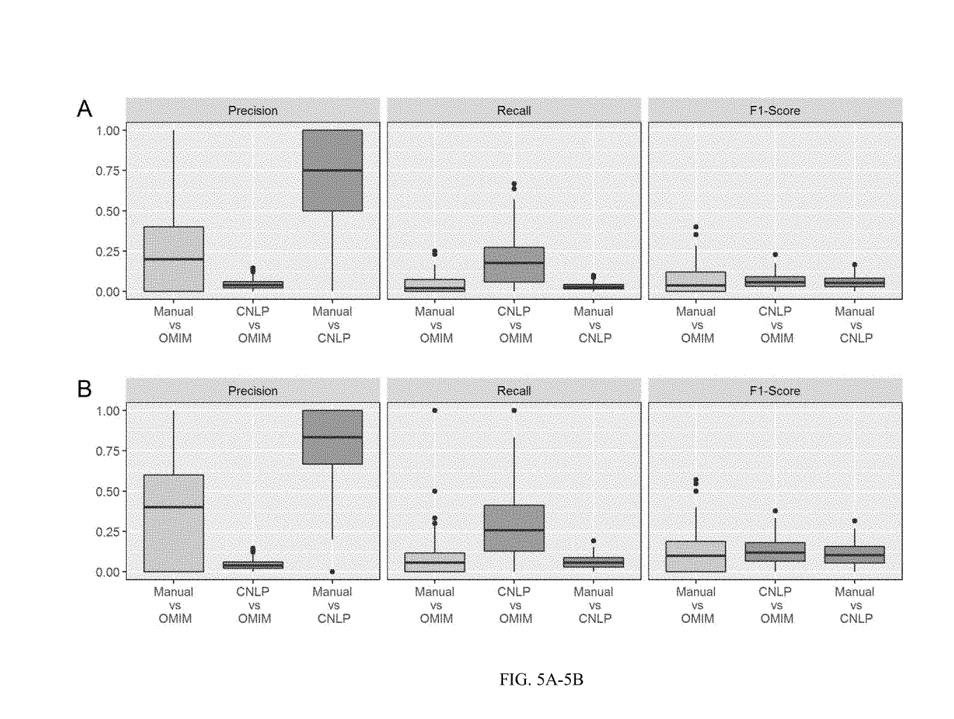

In the 101 diagnosed children, phenotypic features extracted by CNLP overlapped expected OMIM phenotypic features (mean 4.31 terms, SD 4.59, range 0-32) significantly more than the manual extracted phenotypic features (mean 0.92 terms, SD 1.02, range 0-4; P<0.0001, paired Wilcoxon test; FIG. 3B). Although the cohort included eight genetic diseases that were incidental findings, their exclusion did not materially change these results (FIG. 4). Thus, the recall of OMIM phenotypic features by CNLP, although small (mean 0.20, SD 0.16, range 0-0.67), was substantially greater than the sparse expert manual phenotypic features used in expert manual interpretation (mean 0.04, SD 0.06, range 0-0.25) (FIG. 5). However, the much larger number of phenotypic features extracted by CNLP was associated with lower precision (mean 0.04, SD 0.03, range 0-0.15) than manual extraction (mean 0.25, SD 0.30, range 0-1) when compared with OMIM, indicating that, by design, an autonomous diagnostic system should not penalize false positive phenotypic features. Recall and F₁ value increased when phenotypic features with one degree of hierarchical separation to those extracted were included (mean CNLP recall with inexact matches 0.29, SD 0.22, range 0-1; mean CNLP F₁ with inexact matches 0.12, SD 0.08, range 0-0.38; mean CNLP F₁ with exact matches 0.06, SD 0.05, range 0-0.23), indicating that, by design, an autonomous system should include hierarchical parents of extracted terms (FIG. 5).

Traditionally, genetic diseases have been clinically diagnosed by the identification of one or more pathognomonic phenotypic features. Such phenotypic features have high information content (IC, the logarithm of the probability of that phenotypic feature being observed in all OMIM diseases; FIG. 2) (48). A potential concern was that phenotypic features extracted by CNLP would have less information content than those prioritized manually by experts during interpretation. However, among the 101 children, the mean IC of CNLP phenotypic features (8.1, SD 2.0, range 2.6-11.4) was significantly higher than manual (7.8, SD 2.0, range 2.1-11.4; P=0.003, Mann-Whitney U test) or OMIM phenotypic features (7.3, SD 1.7, range 3.2-11.4; P<0.0001, Mann-Whitney U test, FIG. 3E). The inventors note that the mean IC correlated significantly with number of phenotypic features extracted manually and by CNLP (Spearman's rho 0.24, P=0.02 and Spearman's rho 0.44, P<0.0001, respectively; FIG. 3C). The mean IC of CNLP phenotypic features was higher than manual phenotypic features (FIG. 3F), and the mean IC correlated significantly with number of phenotypic features extracted by CNLP (Spearman's rho 0.30, P<0.0001; FIG. 3G).

Retrospective performance of an autonomous system for diagnosis of childhood genetic diseases.

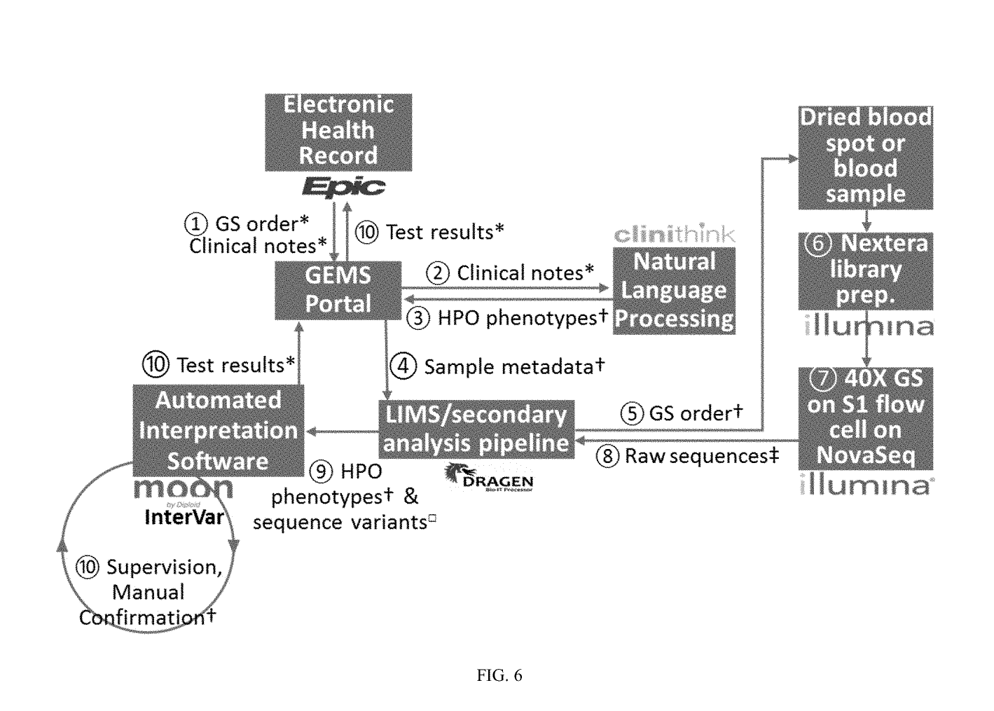

The remaining steps in automated diagnosis of genetic diseases were to combine the automated ranking of the patient's CNLP phenome with respect to all genetic diseases, together with the automated ranking of the pathogenicity of all their genomic variants based on literature knowledge and in silico tools (FIG. 1, FIG. 6). The inventors wrote scripts to transfer the patient's CNLP-derived phenotypic features and genomic variants automatically to autonomous interpretation software (MOON™, Diploid). MOON™ identified the phenotypic features associated with each genetic disease by natural language processing of the medical literature. Typically, this was a larger set of phenotypic features than those listed in the OMIM™ Clinical Synopsis. MOON™ then compared the patient's phenotypic features with those associated with each genetic disease and rank-ordered their likelihood of causing the child's illness.

The inventors also wrote scripts to transfer a patient's nucleotide and structural variants automatically from the DRAGEN™ platform to MOON as soon as it finished, without user intervention. For rapid genome sequencing, there was a mean of 4,742,595 nucleotide variants and 19.3 structural variants (SVs) and exome sequencing had a mean of 39,066 nucleotide variants and 10.3 SVs per patient. Of these, MOON™ retained 67,589 nucleotide variants and 12 SVs, and 791 nucleotide variants and 4.5 SVs, for rapid genome and exome sequencing, respectively, that had allele frequencies <2% and affected known disease genes. A Bayesian framework and probabilistic model in MOON™ ranked the pathogenicity of these variants with 15 in silico prediction tools, ClinVar™ assertions, and inheritance pattern-based allele frequencies. In singleton and family trio analyses, a mean of five and three provisional diagnoses were ranked, respectively (Table 6). Since MOON™ was optimized for sensitivity, it shortlisted a median of 6 nucleotide variants per diagnosed subject (range 2-24), and often shortlisted false positive diagnoses in cases considered negative by manual interpretation. Both were largely remedied, however, by processing the MOON™ output in InterVar™ software, and retaining only pathogenic and likely pathogenic variants (49). InterVar™ classified variants with regard to 18 of the 28 consensus pathogenicity recommendations (50), specifically triaging variants of uncertain significance (VUS). Automated interpretation took a median of five minutes from transfer of variants and HPO terms to display of the provisional diagnosis and supporting evidence, including patient phenotypic features matching that disorder, for laboratory director review. In four timed runs, the time from blood or blood spot receipt to display of the correct diagnosis as the top ranked variant was 19:14-20:25 hours (median 19:38 hours, Table 1, retrospective cases). This conformed well to a daily clinical operation cycle: sample receipt in the morning enabled library preparation in the afternoon, genome sequencing overnight, and provisional reporting early the following morning for laboratory director review.

The inventors retrospectively examined the concordance between the autonomous system and prior, team-based, manual expert interpretation in 95 of the 101 children, diagnosed with 97 of the 105 genetic diseases. The inventors excluded 8 findings that had been reported but that were considered incidental (without current evidence of any of the expected phenotypic features). This cohort was diverse in race and ancestry. Eleven diagnoses were associated with structural variants, and 86 with nucleotide variants. No training patients were included in the test set. In two patients, a revised clinical report was issued of a new diagnosis (infant 6007, EIEE9, Xp22 del, and patient 6033, Cockayne syndrome B, ERCC6 p.Gly528Glu and c.-15+3G>T, which was validated by functional studies). Therefore, initial expert manual interpretation had a recall of 98% (95 of 97). Although the inventors did not re-analyze manual diagnoses, none of them had been demoted in the period since initially reported clinically. The autonomous diagnostic system had precision of 99% (93 of 94) and recall of 97% (94 of 97). For nucleotide and structural variants, the median rank of the correct diagnosis was first (range 1-4 nucleotide variants; range 1-13 SV; Table 6).

The three false negative autonomous diagnoses comprised the following cases.

Infant 6159, with autosomal dominant Alport syndrome (COL4A4 c.4715C>T, p.Pro1572Leu), had hematuria, nephrotic syndrome, glomerulonephritis, hypertension, and anasarca. OMIM™ indicated COL4A4-associated Alport syndrome (CAS) was autosomal recessive, and p.Pro1572Leu was recorded as pathogenic in ClinVar™ for autosomal recessive Alport syndrome. There are, however, a large number of reports of autosomal dominant CAS. The variant was maternally inherited. Since the infant's mother was asymptomatic, the inventors assumed that she exhibited incomplete penetrance of autosomal dominant CAS, as has been reported (51, 52). The autonomous system classified the infant as a carrier for autosomal recessive CAS.

Infant 253 had autosomal dominant optic atrophy plus syndrome (OPA1 c.556+1G>A). The autonomous system did not rank this variant because of insufficient overlap of the 70 CNLP phenotypic features with the MOON™ disease phenotypic feature model. Recent reports indicate that OPA1 can be associated with complex, severe multi-system mitochondrial disorders, similar to infant 253.

Neonate 213 had dextrocardia and transposition of the great vessels. He received singleton genome sequencing, and was diagnosed manually with autosomal dominant visceral heterotaxy type 5 associated with a likely pathogenic variant in NODAL (c.778G>A; p.Gly260Arg). This variant was filtered out by the autonomous system based on classification as a VUS by InterVar™ (based on PM1-PP3-PP5) and the presence of conflicting interpretations in ClinVar, including a ‘Likely Benign’ assertion.

When the relatively sparse phenotypic features selected by experts during manual interpretation were substituted for phenotypic features identified by CNLP, the recall of the autonomous system decreased (88%, 85 of 97).

Prospective Performance of an Autonomous System for Diagnosis of Childhood Genetic Diseases.

The inventors prospectively compared the performance of the autonomous diagnostic system with the fastest manual methods in seven seriously ill infants in intensive care units and three previously diagnosed infants (Table 1). The median time from blood sample to diagnosis with the autonomous platform was 19:56 hours (range 19:10-31:02 hours), compared with the median manual time of 48:23 hours (range 34:38-56:03 hours). This included two automated runs which were delayed by operator error or data center downtime. The autonomous system coupled with InterVar™ post-processing made three diagnoses and no false positive diagnoses. All three diagnoses were confirmed by manual methods and Sanger sequencing. The first was for patient 352, a seven-week-old female, admitted to the pediatric intensive care unit with diabetic ketoacidosis. Rapid genome sequencing was performed on the singleton proband. In 19:11 hours, the autonomous system identified a previously unreported, heterozygous missense variant in the insulin gene (INS c.26C>G, pPro9Arg), which is associated with autosomal dominant permanent neonatal diabetes mellitus (OMIM disease record 606176). According to ACMG/AMP pathogenicity criteria, the variant was of uncertain significance (VUS). After 42:04 hours, parent-child trio sequencing with the fastest manual methods confirmed the result and showed the variant to be de novo, which changed the variant classification to likely pathogenic.

The second diagnosis was made in patient 7052, a previously healthy 17-month-old boy admitted to the pediatric intensive care unit with pseudomonal septic shock, metabolic acidosis, echthyma gangrenosum and hypogammaglobulinemia. Singleton, proband, rapid sequencing and automated interpretation identified a pathogenic hemizygous variant in the Bruton tyrosine kinase gene (BTK c.974+2T>C) associated with X-linked agammaglobulinemia 1 (OMIM: 300755) in 22:04 hours. This was 16:33 hours earlier than a concurrent trio run with the fastest manual methods. The provisional result provided confidence in treatment with high-dose intravenous immunoglobulin (to maintain serum IgG >600 mg/dL) and six weeks of antibiotic treatment. This provisional diagnosis was verbally conveyed to the clinical team upon review of the autonomous result by a laboratory director. Clinical whole genome sequencing subsequently returned the same result and showed the variant to be maternally inherited.

The third diagnosis was made in patient 412, a 3-day-old boy admitted to the neonatal ICU with seizures and a strong family history of infantile seizures responsive to phenobarbital. The autonomous system identified a likely pathogenic, heterozygous variant in the potassium voltage-gated channel, KQT-like subfamily, member 2 gene (KCNQ2 c.1051C>G). This gene is associated with autosomal dominant benign familial neonatal seizures 1 (OMIM™ disease record 121200). The diagnosis was made in 20:53 hours, which was 27:30 hours earlier than a concurrent run with the fastest manual methods. A verbal provisional result was conveyed to the clinical team upon review of the result by a laboratory director as the diagnosis provided confidence in treatment with phenobarbital and changed the prognosis.

For the remaining four patients, no diagnosis was evident with either manual or autonomous methods.

Discussion

Previously, the fastest time to diagnosis by genome sequencing in clinical practice was 37 hours (8, 15-26). The protocol was, however, extremely labor- and capital-intensive, and limited to one sample at a time. Here the inventors described a prototypic, autonomous system for genetic disease diagnosis in a median of 20:10 hours requiring decreased user intervention and a throughput of up to two parent-child trios or six probands per run. Most decision making in ICUs is made deliberatively in morning rounds attended by a multidisciplinary healthcare team. Thus, a 20-hour diagnosis would return results to the on-call physician who ordered testing in time for morning rounds. This would simplify information transfer during rounds and facilitate management decisions. A 20-hour diagnosis is important in seriously ill infants as a majority of timely genomic diagnoses result in changes in ICU management (16-25).

The autonomous platform for 20-hour diagnosis of genetic diseases was designed to meet the needs of acutely ill infants in ICUs with diseases of unknown etiology. It has been estimated that 10-12% of infants admitted to regional ICUs may benefit from same-day diagnosis and implementation of targeted treatments (8, 16-30). In 2014, the US Food and Drug Administration (FDA) permitted provisional reporting in seriously ill children when the diagnosis indicated changes in management that could improve outcome, and where a delay in reporting until confirmation of results by Sanger sequencing could result in avoidable morbidity or mortality (18, 20, 21). In our previous experience, provisional diagnoses were reported in 17% (114 of 684) of genome sequencing cases, with a mean time to report of 3.6 days. Presentations in which 20-hour diagnoses were likely to be associated with improved outcomes included neonatal epileptic encephalopathies, metabolic diseases (as in patient 352), septic shock possibly associated with immunodeficiency (as in patient 7052), organ failure, and when extra-corporeal membrane oxygenation is considered in the absence of a known disease etiology (18-24, 28). Thus, a circumscribed application of an autonomous diagnostic system is to identify provisional diagnoses for laboratory director review, earlier than standard rapid testing, in a subset of neonatal and pediatric ICU admissions in which morbidity or mortality is likely to be avoided by early institution of targeted treatment. It will be important to evaluate the proportion of seriously ill patients and extent of urgent healthcare settings in which a 20-hour diagnosis would inform acute interventions and for which a longer time to result would not be effective.

This disclosure demonstrated the automated extraction of a deep, digital phenome from the EHR. The analytic performance of the extraction of phenotypic features from the EHRs of children with genetic diseases by CNLP herein was considerably better than prior reports, and appeared adequate for replacement of expert manual EHR review (36, 41). CNLP extracted 27-fold more phenotypic features from the EHR than those selected by experts during manual interpretation, consistent with prior reports (36, 41, 47). In addition, the mean information content of the CNLP phenome was greater than that of the phenotypic features selected by experts during manual interpretation. The superiority of deep CNLP phenomes was shown by substantially greater overlap with the expected (OMIM) clinical features than by those selected by experts during manual interpretation. Phenotypic features selected by experts during manual interpretation had poorer diagnostic utility than CNLP-based phenotypic features when used in the autonomous diagnostic system. This concurred with two recent reports of genomic sequencing of cohorts of patients in which the rate of diagnosis was greater when more than fifteen phenotypic features were used at time of interpretation that when one to five were used (53, 54).

Herein the inventors described fully automated interpretation of sequencing results. In 95 seriously ill children, the autonomous system had 97% recall and 99% precision in recapitulating 97 genetic disease diagnoses made by a team of experts. Where the system suggested more than one diagnosis, the median rank of a variant associated with the correct diagnosis was first. The three false negative autonomous results had explanations that either can be addressed by parameter adjustments or were of types that cause assessments of variant pathogenicity to vary between laboratories (55). Prospectively, molecular laboratory directors determined that the autonomous system made correct provisional diagnoses in three of seven seriously ill ICU infants (100% precision and recall) with an average time saving of 22:19 hours. In light of insufficient expert analysts, molecular laboratory directors, medical geneticists and genetic counselors to expand genomic diagnosis to regional ICU infants worldwide, such diagnostic performance was sufficient to suggest several, high throughput clinical applications (31-33). Supervised autonomous systems may provide effective first-tier, provisional diagnoses, allowing valuable cognitive resources to be reserved for unsolved or difficult cases, manual curation of variants, and clinical report generation which includes a summary of medical management literature. Secondly, in the roughly 67% of cases where manual interpretation fails to provide a diagnosis, it is difficult to know when analysis should be considered complete. With further development, autonomous diagnostic systems could provide an independent, objective analysis in such cases. Thirdly, autonomous systems could re-analyze unsolved cases periodically. This is burdensome to perform manually since 250 new gene-disease associations and 9,200 new variant-disease associations are reported annually. However, re-analysis yields up to 8-10% new diagnoses per annum (56-60). Automated re-analysis could include updated CNLP of the EHR, which would useful when the phenotype evolves with time. A known risk of genetic testing is over-treatment as a result of over-diagnosis (61). Periodic, autonomous re-analysis would also detect cases where the diagnosis is changed as a result of reclassification of the causality of the gene or pathogenicity of the variant and/or phenome overlap was minimal. An autonomous system, akin to an autopilot, can decrease the labor intensity of genome interpretation. 106 years after the invention of the autopilot, however, two pilots are still employed in cockpits of commercial aircraft. Likewise, a skilled team will still be required to curate the literature and make tough decisions/classifications for the foreseeable future.

The autonomous system has several limitations. Firstly, system performance is partly predicated on the quality of the history and physical examination, and completeness of the write-up in EHR notes. The performance of the autonomous diagnostic system, though acceptable, is anticipated to improve with additional training, increased mapping of human phenotype ontology terms associated with genetic diseases in OMIM™, Orphanet™ and the literature to SNOMED-CT™, the native language of the CNLP, inclusion of phenotypes from structured EHR fields, measurements of phenotype severity (such as phenotype term frequency in EHR documents), and material negative phenotypes (pathognomonic phenotypes whose absence rules out a specific diagnosis). As part of this, a quantitative data model is needed for improved multivariate matching of non-independent phenotypes that appropriately weights related, inexact phenotype matches. Although possible, the autonomous system did not take advantage of commercial variant database annotations, such as the Human Gene Mutation Database™, and does not eliminate the labor-intensive literature curation which is the current standard for variant reporting. Diagnosis of genetic diseases due to structural variants requires standard library preparation and additional software steps that add several hours to turnaround time. Because the autonomous system utilizes the same knowledge of allele and disease frequencies as manual interpretation, which under-represent minority races or ethnicities, pathogenicity assertions in the latter groups are less certain. Likewise, as the autonomous system utilizes the same consensus guidelines for variant pathogenicity determination as manual interpretation, it is subject to the same general limitations of assertions of pathogenicity (55-61).

The major barriers to widespread adoption of genomic medicine for seriously ill infants with disorders of unknown etiology are an untrained medical workforce and substantial shortage of domain experts, including medical geneticists, molecular laboratory directors and genetic counselors. Manual genome analysis and interpretation are very labor intensive. In addition, the extreme number of rare genetic diseases precludes easy domain mastery by non-experts. Thus, pediatric genomic medicine may be one of the first clinical areas where artificial intelligence is necessary for its general adoption (62). Diagnosis of seriously ill infants with diseases of unknown etiology represents an early application of autonomous diagnostic systems as such cases are abundant in ICUs and a faster time to result is critical for optimal outcomes.

### FIGURE LEGENDS

FIG. 1. Flow diagrams of the diagnosis of genetic diseases by standard and rapid genome sequencing. A. Steps in conventional clinical diagnosis of a single patient by genome sequencing (GS) with manual analysis and interpretation in a minimum of 26 hours, but with mean time-to-diagnosis of sixteen days (8, 16-30). Genome sequencing was requested manually. The inventors extracted genomic DNA manually from blood, assessed DNA quality (QA), and normalized the DNA concentration manually. The inventors then manually prepared TruSeq PCR-free DNA™ sequencing libraries, performed QA again, and normalized the library concentration manually. Genome sequencing was performed on the HiSeg™ 2500 system (Illumina) in rapid run mode (RRM). Sequences were manually transferred to the DRAGEN™ Platform version 1 (Illumina) for alignment and variant calling. Phenotypic features were identified by manual review of the electronic health record (EHR). Variant files and phenotypic features were loaded manually into Opal™ software (Fabric), and interpretation was performed manually. B. Steps in autonomous diagnosis of up to six patients concurrently in a minimum of 19 hours (FIG. 6). Steps included: 1. Automation of order entry from the EHR with a portal; 2. Manual or robotic preparation of Nextera DNA Flex™ sequencing libraries directly from blood in 2.5 hours; 3. Rapid 40-fold coverage genome sequencing in 15.5 hours with the NovaSeq 6000 system and 51 flowcell (Illumina); 4. Automation of sequence transfer, alignment and variant calling in one hour with the DRAGEN platform, version 2 (Illumina); 5. Automated extraction of patient phenomes from the EHR by clinical natural language processing (CNLP), and translation to human phenotype ontology (HPO) terms in 20 seconds; 6. Automated transfer of variant and phenotype files, and automated Bayesian comparison of the CNLP phenome with those of all genetic diseases (MOON, Diploid), combined with automated assessment of the pathogenicity of their genomic variants based on aggregated literature knowledge and in silico predictive tools (InterVar) and automated display of the highest ranked provisional diagnosis(es).

FIG. 2. Clinical natural language processing can extract a more detailed phenome than manual EHR review or OMIM™ clinical synopsis. A. Example CNLP of a sentence from the EHR of an eight-day-old baby (patient 341) with maple syrup urine disease, showing four extracted HPO terms. B. Hierarchical display of HPO phenotypic features extracted by manual review of the EHR of neonate 341, CNLP (red), and expected phenotypic features (from the OMIM Clinical Synopsis, blue). Yellow circles: Phenotypic features extracted by both CNLP and expert review. Purple circles: Phenotypic overlap between CNLP and OMIM™. Grey circles: The location of parent terms of identified phenotypic features within the HPO hierarchy. The Information Content (IC) was defined by IC (phenotype)=−log (p_{phenotype}), where p_{phenotype} was the probability of observing the exact term or one of its subclasses across all diseases in OMIM™. Information content increases from top (general) to bottom (specific).

FIG. 3. Comparison of observed and expected phenotypic features of 375 children with suspected genetic diseases. A-D: 101 children diagnosed with 105 genetic diseases. E-H: 274 children with suspected genetic diseases that were not diagnosed by genomic sequencing. Phenotypic features identified by manual EHR review are in yellow, those identified by CNLP are in red, and the expected phenotypic features, derived from the OMIM™ Clinical Synopsis, are in blue. A. Frequency distribution of the number of phenotypic features (log-transformed) in 101 children with genetic diseases. The mean number of features detected per patient was 4.2 (SD 2.6, range 1-16) for manual review, 116.1 (SD 93.6, range 13-521) for CNLP, and 27.3 (SD 22.8, range 1-100) for OMIM™ (OMIM™ vs Manual: P<0.0001; CNLP vs OMIM™: P<0.0001; CNLP vs Manual: P<0.0001; paired Wilcoxon tests). B. Frequency distribution of information content (IC) for each phenotypic feature set in 101 diagnosed patients. The mean IC was 7.8 (SD 2.0, range 2.1-11.4) for manual review, 8.1 (SD 2.0, range 2.6-11.4) for CNLP, and 7.3 (SD 1.7, range 3.2-11.4) for OMIM™ (Manual vs OMIM™: P<0.0001; CNLP vs OMIM™: P<0.0001; Manual vs CNLP: P=0.003; Mann-Whitney U tests). C. Correlation of the mean information content of phenotypic terms with the number of phenotypic terms in each patient. Spearman's rank correlation coefficient (rₛ) was 0.24 for manually extracted phenotypic features (P=0.02), 0.44 for CNLP (P<0.0001) and −0.001 for OMIM™ (P>0.05). D. Venn diagram showing overlap of phenotypic terms by the three methods for diagnosed patients. Phenotypic features extracted by CNLP overlapped expected OMIM™ phenotypic features (mean 4.31 terms, SD 4.59, range 0-32) significantly more than manually (mean 0.92 terms, SD 1.02, range 0-4; P<0.0001, paired Wilcoxon test for the difference in the number of terms that overlap with OMIM). E. Frequency distribution of the number of phenotypic features (log-transformed) in 274 children with suspected genetic diseases that were not diagnosed by genomic sequencing. The mean number of features was 3.0 (SD 1.9, range 1-12) for manual review and 90.7 (SD 81.1, range 6-482) for CNLP (CNLP vs Manual: P<0.0001, paired Wilcoxon test). F. Frequency distribution IC for each phenotypic feature set in 273 undiagnosed patients. The mean IC was 7.7 (SD 2.1, range 2.1-11.4) for manual review and 8.1 (SD 2.0, range 2.6-11.4) for CNLP (Manual-CNLP: P<0.0001, Mann-Whitney U test). G. Correlation of the mean information content of phenotypic terms with the number of phenotypic terms in each patient. rₛ was 0.02 for manually extracted phenotypic features (P>0.05) and 0.30 for CNLP (P<0.0001). H. Venn diagram showing overlap of phenotypic terms for undiagnosed patients by CNLP and manual methods.

FIG. 4. Venn diagram showing overlap of observed and expected patient phenotypic features in 95 children diagnosed with 97 genetic diseases. Phenotypic features identified by expert manual EHR review during interpretation are shown in yellow. Phenotypic features identified by CNLP are shown in red. The expected phenotypic features are derived from the OMIM™ Clinical Synopsis and are shown in blue. The inventors excluded eight diagnoses that were considered to be incidental findings. Phenotypes extracted by CNLP overlapped expected OMIM™ phenotypes (mean 4.55, SD 4.62, range 0-32) more than phenotypes that were manually extracted (mean 0.97, SD 1.03, range 0-4).

FIG. 5. Precision, recall, and F1-score of phenotypic features identified manually, by CNLP, and OMIM™. Data are from 101 children with 105 genetic diseases. Precision (PPV) was given by tp/tp+fp, where tp were true positives and fp were false positives. Recall (sensitivity) was given by tp/tp+fn, where fn were false negatives. A. Precision and recall calculated based on exact phenotypic feature matches. Manual vs OMIM™—Precision: mean 0.25, SD 0.30, range 0-1; Recall: mean 0.04, SD 0.06, range 0-0.25; F₁: mean 0.07, SD 0.09, range 0-0.40. cNLP vs OMIM—Precision: mean 0.04, SD 0.03, range 0-0.15; Recall: mean 0.20, SD 0.16, range 0-0.67; F₁: mean 0.06, SD 0.05, range 0-0.23. Manual vs cNLP—Precision: mean 0.71, SD 0.28, range 0-1; Recall: mean 0.03, SD 0.02, range 0-0.1; F₁: mean 0.06, SD 0.04, range 0-0.17. B. Precision and recall calculated allowing for inexact phenotype matches (terms with one degree of hierarchical separation). Manual vs OMIM™—Precision: mean 0.4, SD 0.34, range 0-1; Recall: mean 0.09, SD 0.13, range 0-1; F₁: mean 0.13, SD 0.13, range 0-0.57. cNLP vs OMIM™—Precision: mean 0.09, SD 0.07, range 0-0.38; Recall: mean 0.29, SD 0.22, range 0-1; F₁: mean 0.12, SD 0.08, range 0-0.38. Manual vs cNLP—Precision: mean 0.79, SD 0.24, range 0-1; Recall: mean 0.06, SD 0.04, range 0-0.19; F₁: mean 0.11, SD 0.07, range 0-0.32.

FIG. 6. Flow diagram of the software components of the autonomous system for provisional diagnosis of genetic diseases by rapid genome sequencing. Abbreviations: GS: rapid whole genome sequencing; GEMS: Genome management system; HPO: Human Phenotype Ontology; LIMS: Clarity laboratory information management system. Data types were as follows: *: HL7/FHIR; †: JSON; ‡:bcl; □: vcf.

### REFERENCES

1. M. K. Khokha, L. E. Mitchell, J. B. Wallingford, White paper on the study of birth defects. Birth Defects Res 109, 180-185 (2017).
2. The March of Dimes data book for policy makers: maternal, infant and child health in the United States 2016. (2016; http://www.marchofdimes.org/March-of-Dimes-2016-Databook.pdf).
3. S. L. Murphy, J. Xu, K. D. Kochanek, E. Arias, “Mortality in the United States, 2017,” NCHS Data Brief (2018; https://www.ncbi.nlm.nih.gov/pubmed/30500322).
4. P. W. Yoon, R. S. Olney, M. J. Khoury, W. M. Sappenfield, G. F. Chavez, D. Taylor, Contribution of birth defects and genetic diseases to pediatric hospitalizations. A population-based study. Arch Pediatr Adolesc Med 151, 1096-1103 (1997).
5. A. C. Arth, S. C. Tinker, R. M. Simeone, E. C. Ailes, J. D. Cragan, S. D. Grosse, Inpatient Hospitalization Costs Associated with Birth Defects Among Persons of All Ages—United States, 2013. MMWR Morb Mortal Wkly Rep 66, 41-46 (2017).
6. M. A. Berry, P. S. Shah, R. T. Brouillette, J. Hellmann, Predictors of mortality and length of stay for neonates admitted to children's hospital neonatal intensive care units. J Perinatol 28, 297-302 (2008).
7. Committee on Approaching Death: Addressing Key End of Life Issues; Institute of Medicine., in Dying in America: Improving Quality and Honoring Individual Preferences Near the End of Life. (The National Academies Press, Washington (DC), 2015), chap. Appendix F Pediatric End-of-Life and Palliative Care: Epidemiology and Health Service Use.
8. H. Daoud, S. M. Luco, R. Li, E. Bareke, C. Beaulieu, O. Jarinova, N. Carson, S. M. Nikkel, G. E. Graham, J Richer, C. Armour, D. E. Bulman, P. Chakraborty, M. Geraghty, M. A. Lines, T. Lacaze-Masmonteil, J. Majewski, K. M. Boycott, D. A. Dyment, Next-generation sequencing for diagnosis of rare diseases in the neonatal intensive care unit. CMAJ 188, E254-260 (2016).
9. F. Malam, T. Hartley, M. K. Gillespie, C. M. Armour, E. Bariciak, G. E. Graham, S. M. Nikkei, J. Richer, S. L. Sawyer, K. M. Boycott, D. A. Dyment, Benchmarking outcomes in the Neonatal Intensive Care Unit: Cytogenetic and molecular diagnostic rates in a retrospective cohort. Am J Med Genet A 173, 1839-1847 (2017).
10. V. Shashi, A. McConkie-Rosell, B. Rosell, K. Schoch, K. Vellore, M. McDonald, Y. H. Jiang, P. Xie, A. Need, D. B. Goldstein, The utility of the traditional medical genetics diagnostic evaluation in the context of next-generation sequencing for undiagnosed genetic disorders. Genet Med 16, 176-182 (2014).
11. J. Weiner, J. Sharma, J. Lantos, H. Kilbride, How infants die in the neonatal intensive care unit: trends from 1999 through 2008. Arch Pediatr Adolesc Med 165, 630-634 (2011).
12. J. E. Petrikin, L. K. Willig, L. D. Smith, S. F. Kingsmore, Rapid whole genome sequencing and precision neonatology. Semin Perinatol 39, 623-631 (2015).
13. L. D. Smith, L. K. Willig, S. F. Kingsmore, Whole-Exome Sequencing and Whole-Genome Sequencing in Critically Ill Neonates Suspected to Have Single-Gene Disorders. Cold Spring Harb Perspect Med 6, a023168 (2015).
14. “OMIM Entry Statistics,” (Johns Hopkins University, Baltimore, Md., 2018; https://www.omim.org/statistics/geneMap).
15. National Center for Biotechnology Information, National Library of Medicine., Database of Single Nucleotide Polymorphisms (dbSNP). (2018;
   https://www.ncbi.nlm.nih.gov/dbvar?term=(%22clin %20pathogenic%22%5BFilter%5D)%20AND%20homo%20sapiens%5BOrganism%5D).
16. N. A. Miller, E. G. Farrow, M. Gibson, L. K. Willig, G. Twist, B. Yoo, T. Marrs, S. Corder, L. Krivohlavek, A. Walter, J. E. Petrikin, C. J. Saunders, I. Thiffault, S. E. Soden, L. D. Smith, D. L. Dinwiddie, S. Herd, J. A. Cakici, S. Catreux, M. Ruehle, S. F. Kingsmore, A 26-hour system of highly sensitive whole genome sequencing for emergency management of genetic diseases. Genome Med 7, 100 (2015).
17. C. J. Saunders, N. A. Miller, S. E. Soden, D. L. Dinwiddie, A. Noll, N. A. Alnadi, N. Andraws, M. L. Patterson, L. A. Krivohlavek, J. Fellis, S. Humphray, P. Saffrey, Z. Kingsbury, J. C. Weir, J. Betley, R. J. Grocock, E. H. Margulies, E. G. Farrow, M. Artman, N. P. Safina, J. E. Petrikin, K. P. Hall, S. F. Kingsmore, Rapid whole-genome sequencing for genetic disease diagnosis in neonatal intensive care units. Sci Transl Med 4, 154ra135 (2012).
18. L. Farnaes, A. Hildreth, N. M. Sweeney, M. M. Clark, S. Chowdhury, S. Nahas, J. A. Cakici, W. Benson, R. H. Kaplan, R. Kronick, M. N. Bainbridge, J. Friedman, J. J. Gold, Y. Ding, N. Veeraraghavan, D. Dimmock, S. F. Kingsmore, Rapid whole-genome sequencing decreases infant morbidity and cost of hospitalization. npj Genomic Medicine 3, 10 (2018).
19. L. Meng, M. Pammi, A. Saronwala, P. Magoulas, A. R. Ghazi, F. Vetrini, J. Zhang, W. He, A. V. Dharmadhikari, C. Qu, P. Ward, A. Braxton, S. Narayanan, X. Ge, M. J. Tokita, T. Santiago-Sim, H. Dai, T. Chiang, H. Smith, M. S. Azamian, L. Robak, B. L. Bostwick, C. P. Schaaf, L. Potocki, F. Scaglia, C. A. Bacino, N. A. Hanchard, M. F. Wangler, D. Scott, C. Brown, J. Hu, J. W. Belmont, L. C. Burrage, B. H. Graham, V. R. Sutton, W. J. Craigen, S. E. Plon, J. R. Lupski, A. L. Beaudet, R. A. Gibbs, D. M. Muzny, M. J. Miller, X. Wang, M. S. Leduc, R. Xiao, P. Liu, C. Shaw, M. Walkiewicz, W. Bi, F. Xia, B. Lee, C. M. Eng, Y. Yang, S. R. Lalani, Use of Exome Sequencing for Infants in Intensive Care Units: Ascertainment of Severe Single-Gene Disorders and Effect on Medical Management. JAMA Pediatr 171, e173438 (2017).
20. J. E. Petrikin, J. A. Cakici, M. M. Clark, L. K. Willig, N. M. Sweeney, E. G. Farrow, C. J. Saunders, I. Thiffault, N. A. Miller, L. Zellmer, S. M. Herd, A. M. Holmes, S. Batalov, N. Veeraraghavan, L. D. Smith, D. P. Dimmock, J. S. Leeder, S. F. Kingsmore, The NSIGHT1-randomized controlled trial: rapid whole-genome sequencing for accelerated etiologic diagnosis in critically ill infants. NPJ Genom Med 3, 6 (2018).
21. L. K. Willig, J. E. Petrikin, L. D. Smith, C. J. Saunders, I. Thiffault, N. A. Miller, S. E. Soden, J. A. Cakici, S. M. Herd, G. Twist, A. Noll, M. Creed, P. M. Alba, S. L. Carpenter, M. A. Clements, R. T. Fischer, J. A. Hays, H. Kilbride, R. J. McDonough, J. L. Rosterman, S. L. Tsai, L. Zellmer, E. G. Farrow, S. F. Kingsmore, Whole-genome sequencing for identification of Mendelian disorders in critically ill infants: a retrospective analysis of diagnostic and clinical findings. Lancet Respir Med 3, 377-387 (2015).
22. L. Farnaes, S. A. Nahas, S. Chowdhury, J. Nelson, S. Batalov, D. M. Dimmock, S. F. Kingsmore, R. Investigators, Rapid whole-genome sequencing identifies a novel GABRA1 variant associated with West syndrome. Cold Spring Harb Mol Case Stud 3, a001776 (2017).
23. A. Hildreth, K. Wigby, S. Chowdhury, S. Nahas, J. Barea, P. Ordonez, S. Batalov, D. Dimmock, S. Kingsmore, R. Investigators, Rapid whole-genome sequencing identifies a novel homozygous NPC1 variant associated with Niemann-Pick type C1 disease in a 7-week-old male with cholestasis. Cold Spring Harb Mol Case Stud 3, a001966 (2017).
24. E. Sanford, K. Watkins, S. Nahas, M. Gottschalk, N. Coufal, L. Farnaes, D Dimmock, S. Kingsmore, R. Investigators, Rapid whole genome sequencing identifies a novel AIRE variant associated with Autoimmune Polyendocrine Syndrome Type 1. Cold Spring Harb Mol Case Stud 4, a002485 (2018).
25. D. Y. Chen, S. Chowdhury, L. Farnaes, J. R. Friedman, J. Honold, D. P. Dimmock, O. Gold, Rapid Diagnosis of KCNQ2-Associated Early Infantile Epileptic Encephalopathy Improved Outcome. Pediatr Neurol 86, 69-70 (2018).
26. Z. Stark, S. Lunke, G. R. Brett, N. B. Tan, R. Stapleton, S. Kumble, A. Yeung, D. G. Phelan, B. Chong, M. Fanjul-Fernandez, J. E. Marum, M. Hunter, A. Jarmolowicz, Y. Prawer, J. R. Riseley, M. Regan, J. Elliott, M. Martyn, S. Best, T. Y. Tan, C. L. Gaff, S. M. White, Meeting the challenges of implementing rapid genomic testing in acute pediatric care. Genet Med 20, 1554-1563 (2018).
27. L. Mestek-Boukhibar, E. Clement, W. D. Jones, S. Drury, L. Ocaka, A. Gagunashvili, P. Le Quesne Stabej, C. Bacchelli, N. Jani, S. Rahman, L. Jenkins, J. A. Hurst, M. Bitner-Glindzicz, M. Peters, P. L. Beales, H. J. Williams, Rapid Paediatric Sequencing (RaPS): comprehensive real-life workflow for rapid diagnosis of critically ill children. J Med Genet 55, 721-728 (2018).
28. E. Sanford, L. Farnaes, S. Batalov, M. Bainbridge, S. Laubach, H. M. Worthen, M. Tokita, S. F. Kingsmore, J. Bradley, Concomitant diagnosis of immune deficiency and Pseudomonas sepsis in a 19 month old with ecthyma gangrenosum by host whole-genome sequencing. Cold Spring Harb Mol Case Stud 4, a003244 (2018).
29. S. E. Soden, C. J. Saunders, L. K. Willig, E. G. Farrow, L. D. Smith, J. E. Petrikin, J. B. LePichon, N. A. Miller, I. Thiffault, D. L. Dinwiddie, G. Twist, A. Noll, B. A. Heese, L. Zellmer, A. M. Atherton, A. T. Abdelmoity, N. Safina, S. S. Nyp, B. Zuccarelli, I. A. Larson, A. Modrcin, S. Herd, M. Creed, Z. Ye, X. Yuan, R. A. Brodsky, S. F. Kingsmore, Effectiveness of exome and genome sequencing guided by acuity of illness for diagnosis of neurodevelopmental disorders. Sci Transl Med 6, 265ra168 (2014).
30. B. Briggs, K. N. James, S. Chowdhury, C. Thornburg, L. Farnaes, D. Dimmock, S. F. Kingsmore, R. Investigators, Novel Factor XIII variant identified through whole-genome sequencing in a child with intracranial hemorrhage. Cold Spring Harb Mol Case Stud 4, a003525 (2018).
31. Z. Stark, L. Dolman, T. A. Manolio, B. Ozenberger, S. L. Hill, M. J. Caulfied, Y. Levy, D. Glazer, J. Wilson, M. Lawler, T. Boughtwood, J. Braithwaite, P. Goodhand, E. Birney, K. N. North, Integrating Genomics into Healthcare: A Global Responsibility. Am J Hum Genet 104, 13-20 (2019).
32. J. M. Friedman, Y. Bombard, M. C. Cornel, C. V. Fernandez, A. K. Junker, S. E. Plon, Z. Stark, B. M. Knoppers, G. Paediatric Task Team of the Global Alliance for, R. Health, S. Ethics Work, Genome-wide sequencing in acutely ill infants: genomic medicine's critical application? Genet Med 21, 498-504 (2018).
33. A. Borghesi, M. A. Mencarelli, L. Memo, G. B. Ferrero, A. Bartuli, M. Genuardi, M. Stronati, A. Villani, A. Renieri, G. Corsello, S. their respective Scientific, Intersociety policy statement on the use of whole-exome sequencing in the critically ill newborn infant. Ital J Pediatr 43, 100 (2017).
34. U.K. Department of Health and Social Care, Matt Hancock announces ambition to map 5 million genomes. (2018; https://www.gov.uk/government/news/matt-hancock-announces-ambition-to-map-5-million-genomes).
35. Illumina Inc., Nextera DNA Flex Library Prep Reference Guide (Document #1000000025416 v00, 2017; https://support.illumina.com/content/dam/illumina-support/documents/documentation/chemistry_documentation/samplepreps_nextera/nextera_dna_flex/nextera-dna-flex-library-prep-reference-guide-1000000025416-00.pdf).
36. J. H. Son, G. Xie, C. Yuan, L. Ena, Z. Li, A. Goldstein, L. Huang, L. Wang, F. Shen, H. Liu, K. Mehl, E. E. Groopman, M. Marasa, K. Kiryluk, A. G. Gharavi, W. K. Chung, G. Hripcsak, C. Friedman, C. Weng, K. Wang, Deep Phenotyping on Electronic Health Records Facilitates Genetic Diagnosis by Clinical Exomes. Am J Hum Genet 103, 58-73 (2018).
37. G. Hripcsak, D. J. Albers, High-fidelity phenotyping: richness and freedom from bias. J Am Med Inform Assoc 25, 289-294 (2017).
38. W. Q. Wei, J. C. Denny, Extracting research-quality phenotypes from electronic health records to support precision medicine. Genome Med 7, 41 (2015).
39. B. Campillo-Gimenez, N. Garcelon, P. Jarno, J. M. Chapplain, M. Cuggia, Full-text automated detection of surgical site infections secondary to neurosurgery in Rennes, France. Stud Health Technol Inform 192, 572-575 (2013).
40. F. Dhombres, O. Bodenreider, Interoperability between phenotypes in research and healthcare terminologies—Investigating partial mappings between HPO and SNOMED CT. J Biomed Semantics 7, 3 (2016).
41. H. L. Mandel, “Performance evaluation of a natural language processing tool to extract infectious disease problems” thesis, University of Washington Seattle, Wash. (2013 http://bime.uw.edu/wordpress/wp-content/uplo ads/2016/11/Mandel-Hannah-L.-2013-MS.pdf).
42. N. Garcelon, A. Neuraz, R. Salomon, H. Faour, V. Benoit, A. Delapalme, A. Munnich, A. Burgun, B. Rance, A clinician friendly data warehouse oriented toward narrative reports: Dr. Warehouse. J Biomed Inform 80, 52-63 (2018).
43. N. Garcelon, A. Neuraz, V. Benoit, R. Salomon, S. Kracker, F. Suarez, N. Bahi-Buisson, S. Hadj-Rabia, A. Fischer, A. Munnich, A. Burgun, Finding patients using similarity measures in a rare diseases-oriented clinical data warehouse: Dr. Warehouse and the needle in the needle stack. J Biomed Inform 73, 51-61 (2017).
44. G. Carlsson, Topology and data. Bull Am Mathematical Soc 46, 255-308 (2009).
45. S. Kohler, M. H. Schulz, P. Krawitz, S. Bauer, S. Dolken, C. E. Ott, C. Mundlos, D. Horn, S. Mundlos, P. N. Robinson, Clinical diagnostics in human genetics with semantic similarity searches in ontologies. Am J Hum Genet 85, 457-464 (2009).
46. The Human Phenotype Ontology, Build #1246 (2017; http://compbio.charite.de/jenkins/job/hpo.annotations/1246/).
47. N. Garcelon, A. Neuraz, R. Salomon, N. Bahi-Buisson, J. Amiel, C. Picard, N. Mahlaoui, V. Benoit, A. Burgun, B. Rance, Next generation phenotyping using narrative reports in a rare disease clinical data warehouse. Orphanet J Rare Dis 13, 85 (2018).
48. P. Resnik, Semantic similarity in a taxonomy: an information-based measure and its application to problems of ambiguity in natural language. J. Artif. Int. Res. 11, 95-130 (1999).
49. 49. Q. Li, K. Wang, InterVar: Clinical Interpretation of Genetic Variants by the 2015 ACMG-AMP Guidelines. Am J Hum Genet 100, 267-280 (2017).
50. S. Richards, N. Aziz, S. Bale, D. Bick, S. Das, J. Gastier-Foster, W. W. Grody, M. Hegde, E. Lyon, E. Spector, K. Voelkerding, H. L. Rehm, A. L. Q. A. Committee, Standards and guidelines for the interpretation of sequence variants: a joint consensus recommendation of the American College of Medical Genetics and Genomics and the Association for Molecular Pathology. Genet Med 17, 405-424 (2015).
51. M. Kharrat, S. Makni, K. Makni, K. Kammoun, K. Charfeddine, H. Azaeiz, F. Jarraya, M. Ben Hmida, M. C. Gubler, H. Ayadi, J. Hachicha, Autosomal dominant Alport's syndrome: study of a large Tunisian family Saudi J Kidney Dis Transpl 17, 320-325 (2006).
52. C. Pescucci, F. Mari, I. Longo, P. Vogiatzi, R. Caselli, E. Scala, C. Abaterusso, R. Gusmano, M. Seri, N. Miglietti, E. Bresin, A. Renieri, Autosomal-dominant Alport syndrome: natural history of a disease due to COL4A3 or COL4A4 gene. Kidney Int 65, 1598-1603 (2004).
53. M. M. Clark, Z. Stark, L. Farnaes, T. Y. Tan, S. M. White, D. Dimmock, S. F. Kingsmore, Meta-analysis of the diagnostic and clinical utility of genome and exome sequencing and chromosomal microarray in children with suspected genetic diseases. NPJ Genom Med 3, 16 (2018).
54. D. Trujillano, A. M. Bertoli-Avella, K. Kumar Kandaswamy, M. E. Weiss, J. Koster, A. Marais, O. Paknia, R. Schroder, J. M. Garcia-Aznar, M. Werber, O. Brandau, M. Calvo Del Castillo, C. Baldi, K. Wessel, S. Kishore, N. Nahavandi, W. Eyaid, M. T. Al Rifai, A. Al-Rumayyan, W. Al-Twaijri, A. Alothaim, A. Alhashem, N. Al-Sannaa, M. Al-Balwi, M. Alfadhel, A. Rolfs, R. Abou Jamra, Clinical exome sequencing: results from 2819 samples reflecting 1000 families Eur J Hum Genet 25, 176-182 (2017).
55. L. M. Amendola, G. P. Jarvik, M. C. Leo, H. M. McLaughlin, Y. Akkari, M. D. Amaral, J. S. Berg, S. Biswas, K. M. Bowling, L. K. Conlin, G. M. Cooper, M. O. Dorschner, M. C. Dulik, A. A. Ghazani, R. Ghosh, R. C. Green, R. Hart, C. Horton, J. J. Johnston, M. S. Lebo, A. Milosavljevic, J. Ou, C. M. Pak, R. Y. Patel, S. Punj, C. S. Richards, J. Salama, N. T. Strande, Y. Yang, S. E. Plon, L. G. Biesecker, H. L. Rehm, Performance of ACMG-AMP Variant-Interpretation Guidelines among Nine Laboratories in the Clinical Sequencing Exploratory Research Consortium. Am J Hum Genet 98, 1067-1076 (2016).
56. A. M. Wenger, H. Guturu, J. A. Bernstein, G. Bejerano, Systematic reanalysis of clinical exome data yields additional diagnoses: implications for providers. Genet Med 19, 209-214 (2017).
57. E. Williams, K. Retterer, M. Cho, G. Richard, J. Juusola, paper presented at the ACMG 2016, Tampa, Fla., 2016.
58. G. Costain, R. Jobling, S. Walker, M. S. Reuter, M. Snell, S. Bowdin, R. D. Cohn, L. Dupuis, S. Hewson, S. Mercimek-Andrews, C. Shuman, N. Sondheimer, R. Weksberg, G. Yoon, M. S. Meyn, D. J. Stavropoulos, S. W. Scherer, R. Mendoza-Londono, C. R. Marshall, Periodic reanalysis of whole-genome sequencing data enhances the diagnostic advantage over standard clinical genetic testing. Eur J Hum Genet 26, 740-744 (2018).
59. S. Nambot, J. Thevenon, P. Kuentz, Y. Duffourd, E. Tisserant, A. L. Bruel, A. L. Mosca-Boidron, A. Masurel-Paulet, D. Lehalle, N. Jean-Marcais, M. Lefebvre, P. Vabres, S. El Chehadeh-Djebbar, C. Philippe, F. Tran Mau-Them, J. St-Onge, T. Jouan, M. Chevarin, C. Poe, V. Carmignac, A. Vitobello, P. Callier, J. B. Riviere, L. Faivre, C. Thauvin-Robinet, G. Orphanomix Physicians, Clinical whole-exome sequencing for the diagnosis of rare disorders with congenital anomalies and/or intellectual disability: substantial interest of prospective annual reanalysis. Genet Med 20, 645-654 (2017).
60. C. F. Wright, J. F. McRae, S. Clayton, G. Gallone, S. Aitken, T. W. FitzGerald, P. Jones, E. Prigmore, D. Rajan, J. Lord, A. Sifrim, R. Kelsell, M. J. Parker, J. C. Barrett, M. E. Hurles, D. R. FitzPatrick, H. V. Firth, Making new genetic diagnoses with old data: iterative reanalysis and reporting from genome-wide data in 1,133 families with developmental disorders. Genet Med 20, 1216-1223 (2018).
61. A. K. Manrai, B. H. Funke, H. L. Rehm, M. S. Olesen, B. A. Maron, P. Szolovits, D. M. Margulies, J. Loscalzo, I. S. Kohane, Genetic Misdiagnoses and the Potential for Health Disparities. N Engl J Med 375, 655-665 (2016).
62. H. Liang, B. Y. Tsui, H. Ni, C. C. S. Valentim, S. L. Baxter, G. Liu, W. Cai, D. S. Kermany, X. Sun, J. Chen, L. He, J. Zhu, P. Tian, H. Shao, L. Zheng, R. Hou, S. Hewett, G. Li, P. Liang, X. Zang, Z. Zhang, L. Pan, H. Cai, R. Ling, S. Li, Y. Cui, S. Tang, H. Ye, X. Huang, W. He, W. Liang, Q. Zhang, J. Jiang, W. Yu, J. Gao, W. Ou, Y. Deng, Q. Hou, B. Wang, C. Yao, Y. Liang, S. Zhang, Y. Duan, R. Zhang, S. Gibson, C. L. Zhang, O. Li, E. D. Zhang, G. Karin, N. Nguyen, X. Wu, C. Wen, J. Xu, W. Xu, B. Wang, W. Wang, J. Li, B. Pizzato, C. Bao, D. Xiang, W. He, S. He, Y. Zhou, W. Haw, M. Goldbaum, A. Tremoulet, C. N. Hsu, H. Carter, L. Zhu, K. Zhang, H. Xia, Evaluation and accurate diagnoses of pediatric diseases using artificial intelligence. Nat Med. 10.1038/s41591-018-0335-9 (2019).
63. S. Kohler, N. A. Vasilevsky, M. Engelstad, E. Foster, J. McMurry, S. Ayme, G. Baynam, S. M. Bello, C. F. Boerkoel, K. M. Boycott, M. Brudno, O. J. Buske, P F Chinnery, V. Cipriani, L. E. Connell, H. J. Dawkins, L. E. DeMare, A. D. Devereau, B. B. de Vries, H. V. Firth, K. Freson, D. Greene, A. Hamosh, I. Helbig, C. Hum, J. A. Jahn, R. James, R. Krause, F. L. SJ, H. Lochmuller, G. J. Lyon, S. Ogishima, A. Olry, W. H. Ouwehand, N. Pontikos, A. Rath, F. Schaefer, R. H. Scott, M. Segal, P. I. Sergouniotis, R. Sever, C. L. Smith, V. Straub, R. Thompson, C. Turner, E. Turro, M. W. Veltman, T. Vulliamy, J. Yu, J. von Ziegenweidt, A. Zankl, S. Zuchner, T. Zemojtel, J. O. Jacobsen, T. Groza, D. Smedley, C. J. Mungall, M. Haendel, P. N. Robinson, The Human Phenotype Ontology in 2017. Nucleic Acids Res 45, D865-D876 (2017).
64. Z. Powis, A. Hart, S. Cherny, I. Petrik, E. Palmaer, S. Tang, C. Jones, Clinical diagnostic exome evaluation for an infant with a lethal disorder: genetic diagnosis of TARP syndrome and expansion of the phenotype in a patient with a newly reported RBM10 alteration. BMC Med Genet 18, 60 (2017).
65. A. Abyzov, A. E. Urban, M. Snyder, M. Gerstein, CNVnator: an approach to discover, genotype, and characterize typical and atypical CNVs from family and population genome sequencing. Genome Res 21, 974-984 (2011).
66. X. Chen, O. Schulz-Trieglaff, R. Shaw, B. Barnes, F. Schlesinger, M. Kallberg, A. J. Cox, S. Kruglyak, C. T. Saunders, Manta: rapid detection of structural variants and indels for germline and cancer sequencing applications. Bioinformatics 32, 1220-1222 (2016).
67. E. M. Coonrod, R. L. Margraf, A. Russell, K. V. Voelkerding, M. G. Reese, Clinical analysis of genome next-generation sequencing data using the Omicia platform. Expert Rev Mol Diagn 13, 529-540 (2013).
68. H. Hu, C. D. Huff, B. Moore, S. Flygare, M. G. Reese, M. Yandell, VAAST 2.0: improved variant classification and disease-gene identification using a conservation-controlled amino acid substitution matrix. Genet Epidemiol 37, 622-634 (2013).
69. H. Hu, J. C. Roach, H. Coon, S. L. Guthery, K. V. Voelkerding, R. L. Margraf, J. D. Durtschi, S. V. Tavtigian, Shankaracharya, W. Wu, P. Scheet, S. Wang, J. Xing, G. Glusman, R. Hubley, H. Li, V. Garg, B. Moore, L. Hood, D. J. Galas, D. Srivastava, M. G. Reese, L. B. Jorde, M. Yandell, C. D. Huff, A unified test of linkage analysis and rare-variant association for analysis of pedigree sequence data. Nat Biotechnol 32, 663-669 (2014).
70. M. V. Singleton, S. L. Guthery, K. V. Voelkerding, K. Chen, B. Kennedy, R. L. Margraf, J. Durtschi, K. Eilbeck, M. G. Reese, L. B. Jorde, C. D. Huff, M. Yandell, Phevor combines multiple biomedical ontologies for accurate identification of disease-causing alleles in single individuals and small nuclear families Am J Hum Genet 94, 599-610 (2014).
71. K. J. Karczewski, B. Weisburd, B. Thomas, M. Solomonson, D. M. Ruderfer, D. Kavanagh, T. Hamamsy, M. Lek, K. E. Samocha, B. B. Cummings, D. Birnbaum, C. The Exome Aggregation, M. J. Daly, D. G. MacArthur, The ExAC browser: displaying reference data information from over 60 000 exomes. Nucleic Acids Res 45, D840-D845 (2017).
72. Lumaka A, Race V, Peeters H, Corveleyn A, Coban-Akdemir Z, Jhangiani S N, Song X, Mubungu G, Posey J, Lupski J R, Vermeesch J R, Lukusa P, Devriendt K. A comprehensive clinical and genetic study in 127 patients with ID in Kinshasa, D R Congo. Am J Med Genet A. 2018 September; 176(9):1897-1909.
73. R Development Core Team, R: A language and environment for statistical computing. (2017; https://www.R-project.org/).
74. D. Greene, S. Richardson, E. Turro, ontologyX: a suite of R packages for working with ontological data. Bioinformatics 33, 1104-1106 (2017).
75. D. Greene, hpoPlot: Functions for Plotting HPO Terms. R package version 2.4 (2015; https://CRAN.R-project.org/package=hpoPlot).
76. H. Wickham, ggplot2: Elegant Graphics for Data Analysis. (Springer Publishing Company, Incorporated, 2009), pp. 216.
77. J. Larsson, eulerr: Area-Proportional Euler and Venn Diagrams with Ellipses. R package version 4.0.0 (2018; https://cran.r-project.org/package=eulerr).
78. J. M. Zook, B. Chapman, J. Wang, D. Mittelman, O. Hofmann, W. Hide, M. Salit, Integrating human sequence data sets provides a resource of benchmark SNP and indel genotype calls. Nat Biotechnol 32, 246-251 (2014).

### Supplementary Materials

### Tables

TABLE 1
Duration and metrics for the major steps in the diagnosis of genetic diseases by genome sequencing using rapid standard methods (Std.) and a rapid, autonomous platform (Auto.).

| | Use Type | | | | | | | | | | | | | | | | | |
|---|---|---|---|---|---|---|---|---|---|---|---|---|---|---|---|---|---|---|
| | Retrospective Patients | | | | Prospective Patients | | | | | | | | | | | | | |
| Subject ID | 263 | | 6124 | 3003 | 6194 | | 290 | | 352 | | 362 | | 374 | | 7052 | | 412 | |
| Age | 8 days | | 14 years | 1 year | 5 days | | 3 days | | 7 weeks | | 4 weeks | | 2 days | | 17 months | | 3 days | |
| Sex | ♀ | | ♂ | ♀ | ♀ | | ♂ | | ♀ | | ♂ | | ♂ | | ♂ | | ♂ | |
| Abbreviated | Neonatal | | Rhabdo- | Dystonia, | Hypo- | | Pulmonary | | Diabetic | | Neonatal | | HIE | | Pseudo- | | Neonatal | |
| Presentation | seizures | | myolysis | Dev. | glycemia, | | hemorrhage, | | keto- | | seizures | | anemia | | monal | | seizures | |
| | | | | delay | seizures | | PPHN | | acidosis | | | | | | septic | | | |
| | | | | | | | | | | | | | | | shock | | | |
| Method | Auto. | Auto. | Auto. | Auto. | Auto. | Std. | Auto. | Std. | Auto. | Std. | Auto. | Std. | Auto. | Std. | Auto. | Std. | Auto. | Std. |
| Number of | 51 | | 115 | 148 | 14 | 2 | 257 | 4 | 103 | 4 | 65 | 1 | 112 | 6 | 124 | 3 | 33 | 1 |
| Phenotypic | | | | | | | | | | | | | | | | | | |
| Features | | | | | | | | | | | | | | | | | | |
| Molecular | Early | | Glycogen | Dopa- | None | None | None | None | Permanent | | None | None | None | None | X-linked | | Benign | |
| Diagrosis | Infantile | | Storge | Responsive | | | | | neonatal | | | | | | agamma- | | familial | |
| | Epileptic | | Disease | Dystonia | | | | | diabetes | | | | | | globul- | | neonatal | |
| | Encephal- | | V | | | | | | mellitus | | | | | | inemia | | seizures | |
| | opathy 7 | | | | | | | | | | | | | | 1 | | 1 | |
| Gene and | KCNQ2 | | PYGM | TH c.785C > G | n.a. | n.a. | n.a. | n.a. | INS c.26C > G | | n.a. | n.a. | n.a. | n.a. | BTK c.974 + | | KCNQ2 | |
| Causative | c.727C > G | | c.2262delA | c.541C > T | | | | | | | | | | | 2T > C | | c.1051C > G | |
| Variant(s) | | | c.1726C > T | | | | | | | | | | | | | | | |
| Sample/Library | 3:20 | 2:55 | 2:24 | 2:22 | 2:10 | 23:54 | 2:12 | 22:05 | 2:13 | 15:42 | 2:31 | 18:30 | 3:30 | 10:10 | 4:30 | 12:10 | 3:05 | 23:50 |
| Prep (hours) | | | | | | | | | | | | | | | | | | |
| NovaSeq | 0:20 | 0:17 | 0:16 | 0:20 | 1:38* | 0:20 | 0:29 | 0:22 | 0:30 | 0:53 | 0:15 | 2:30 | 0:45 | 0:35 | 1:00 | 1:00 | 0:20 | 0:53 |
| Loading (hours) | | | | | | | | | | | | | | | | | | |
| 2 × 101 nt | 15:36 | 15.31 | 15:34 | 15:27 | 15:26 | 24:13 | 15:25 | 24:08 | 15:21 | 22:44 | 15:17 | 33:36 | 15:17 | 21:07 | 15:19 | 22:46 | 15:58 | 21:00 |
| Sequencing | | | | | | | | | | | | | | | | | | |
| (hours) | | | | | | | | | | | | | | | | | | |
| 1° & 2° Analysis | 1:03 | 1:02 | 0:59 | 0:59 | 1:07 | 3:05 | 1:00 | 1:57 | 1:01 | 2:30 | 1:02 | 2:30 | 1:02 | 2:30 | 1:09 | 2:25 | 1:24 | 2:24 |
| (hours) | | | | | | | | | | | | | | | | | | |
| 3° Analysis | 0:06 | 0:05 | 0:07 | 0:05 | 0:06 | 0:15 | 0:08 | 0:14 | 0:06 | 0:15 | 0:05 | 0:15 | 10:28^{†} | 0:16 | 0:06 | 0:16 | 0:06 | 0:16 |
| Processing | | | | | | | | | | | | | | | | | | |
| (hours) | | | | | | | | | | | | | | | | | | |
| Total (hours) | 20:25 | 19:56 | 19:20 | 19:14 | 20:42* | 56:03 | 19:29 | 48:46 | 19:11 | 42:04 | 19:10 | 57:21 | 31:02^{†} | 34:38 | 22:04 | 38:37 | 20:53 | 48:23 |

Primary (1°) and secondary (2°) Analysis: conversion of raw data from base call to FASTQ format, read alignment to the reference genomes and variant calling.
Tertiary (3°) Analysis Processing: Time to process variants and phenotypic features and make them available for manual interpretation in Opal interpretation software (Fabric Genomics) or to display a provisional, automated diagnosis(es) in MOON interpretation software (Diploid).
Dev. Delay: global developmental delay.
PPHN: Persistent pulmonary hypertension of the newborn.
HIE: Hypoxic ischemic encephalopathy.
n.a.: not applicable.
*Included time to thaw a second set of NovaSeq reagents.
^{†}Included 10:20 hours of downtime, with manual restarting of the job, due to data center relocation.
Patients 263, 6124 and 3003 were retrospectively analyzed by the autonomous system.
Patient 263 was analyzed two times by the autonomous system.
Patients 6194, 290, 352, 362, 412, and 7072 were prospectively analyzed by both autonomous and standard diagnostic methods.

TABLE 2
Comparison of the analytic performance of standard and new library
preparation, and standard and rapid genome sequencing in retrospective samples. The
standard library preparation and genome sequencing methods were TruSeq ™ PCR-free
library preparation and 2 × 100 nt sequencing on a NovaSeq ™ 6000 with S2 flow cell,
respectively. The new library preparation and genome sequencing methods were Nextera
Flex ™ library preparation and 2 × 100 nt sequencing on a NovaSeq ™ 6000 with S1 flow cell,
respectively. The “Median” column is the median of runs R17AA978, R17AA978,
R17AA059, and R17AA119. Controls 1 and 2 are mean values for five and fifty-two samples,
respectively. Analytic performance of variant calls was assessed in sample NA12878, with
comparison to the NIST Genome-in-a-bottle results (76). Note: The NA12878 control run
with the S1 flowcell and TruSeq ™ PCR free library (far right) was 2 × 151 nt.

| | Run | | | | | | | | |
|---|---|---|---|---|---|---|---|---|---|
| | R17AA978 | R17AA978 | R17AA059 | R17AA119 | Median | NA12878 | Control 1 | Control 2 | NA12878 |
| NovaSeq ™ | S1 | | | | S1 | | S2 | S2 | S1 |
| 6000 Flowcell | | | | | | | | | |
| Library | Nextera ™ Flex | | | | Nextera ™ Flex | | Nextera ™ | TruSeq ™ | PCR-free |
| Preparation | | | | | | | Flex | | |
| Method | | | | | | | | | |
| Sample | 263 | 263 | 6124 | 3003 | 263 × 2, | 1 sample | 5 samples | 52 samples | 1 sample |
| | | | | | 6124, 3003 | | | | |
| Raw Yield | 416 | 419 | 404 | 432 | 418 | 435 | 933 | 897 | 537 |
| Per Flowcell | | | | | | | | | |
| (Gb) | | | | | | | | | |
| % Reads Q > 30 | 92.00% | 92.07% | 92.11% | 94.84% | 92.09% | 90.69% | 91.50% | 91.70% | 91.96% |
| Trimmed Yield (Gb) | 153.9 | 158.9 | 165.0 | 160.7 | 159.8 | 148.9 | 183.3 | 152.8 | 164.5 |
| % Reads Mapped | 97.9% | 97.9% | 98.1% | 96.9% | 97.9% | 98.9% | 98.6% | 98.7% | 98.8% |
| % Duplicate Reads | 9.3% | 10.4% | 7.6% | 19.1% | 9.8% | 8.50% | 11.4% | 6.3% | 17.2% |
| Mean Insert Size | 386.0 | 348.0 | 336.0 | 274.0 | 342.0 | 345.1 | 315.1 | 423.4 | 514.6 |
| (nt) | | | | | | | | | |
| Average genome | 42.0 | 43.0 | 44.4 | 39.0 | 42.5 | 47.5 | 49.4 | 43.6 | 32.9 |
| coverage | | | | | | | | | |
| % OMIM genes | 96.0% | 95.7% | 94.9% | 65.1% | 95.3% | 95.8% | 96.8% | 97.7% | 98.00% |
| with 100% | | | | | | | | | |
| coverage at | | | | | | | | | |
| ≥10X | | | | | | | | | |
| Variants | 4,910,055 | 4,915,843 | 4,847,506 | 4,655,831 | 4,878,781 | 4,733,000 | 4,976,974 | 4,922,188 | 4,747,231 |
| Variants passing QC | 96.0% | 96.1% | 96.6% | 96.8% | 96.3% | 96.8% | 98.1% | 98.4% | 98.5% |
| CD Variants | 0.53% | 0.53% | 0.55% | 0.54% | 0.53% | 0.58% | 0.53% | 0.53% | 0.58% |
| Indels | 17.8% | 17.9% | 18.0% | 17.5% | 17.8% | 17.5% | 18.6% | 18.8% | 19.4% |
| CD Homozygous/ | 0.59 | 0.59 | 0.57 | 0.60 | 0.59 | 0.60 | 0.56 | 0.59 | 0.60 |
| Heterozygous | | | | | | | | | |
| Variant | | | | | | | | | |
| Ratio | | | | | | | | | |
| Ti/Tv ratio | 2.02 | 2.02 | 2.02 | 2.03 | 2.02 | 2.02 | 2.02 | 2.02 | 2.01 |
| CD Ti/Tv ratio | 2.85 | 2.87 | 2.88 | 2.94 | 2.88 | 2.81 | 2.85 | 2.85 | 2.82 |
| Analytic | | | | | | | | | |
| Performance | | | | | | | | | |
| PPV (SNV) | n.a. | n.a. | n.a. | n.a. | n.a. | 99.8% | 99.8% | 99.9% | 99.9% |
| PPV (indels) | n.a. | n.a. | n.a. | n.a. | n.a. | 99.0% | 97.0% | 99.3% | 99.7% |
| Sensitivity (SNV) | n.a. | n.a. | n.a. | n.a. | n.a. | 99.7% | 99.6% | 99.7% | 99.8% |
| Sensitivity (indels) | n.a. | n.a. | n.a. | n.a. | n.a. | 95.5% | 96.3% | 99.0% | 99.4% |

Abbreviations:
nt: Nucleotides;
FC: flowcell;
Gb: gigabase;
Q: Quality score;
OMIM: Online Mendelian Inheritance in Man;
QC: Quality Control;
CD: Coding Domain;
Ti/Tv ratio: ratio of the number of nucleotide transitions to the number of nucleotide transversions;
PPV: Positive predictive value;
SNV: single nucleotide variants;
indels: nucleotide insertion-deletion variants.

TABLE 3
Comparison of the analytic performance of standard and new library
preparation and genome sequencing methods in seven matched prospective samples. The
standard library preparation and genome sequencing methods were TruSeq ™ PCR-free
library preparation and NovaSeq 6000 with S2 flow cell, respectively, with the exception of
subjects 7052 and 412, where the library preparation was done with the KAPA Hyper ™ kit.
The new library preparation and genome sequencing methods were Nextera ™ Flex library
preparation and NovaSeq ™ 6000 with S1 flow cell, respectively.

| | Run | | | | | | | |
|---|---|---|---|---|---|---|---|---|
| | R18AA202 | Std. | R18AA218 | Std. | R18AA922 | Std | R18AB113 | Std |
| | Subject | | | | | | | |
| | 6194 (Prospective) | | 290 (Prospective) | | 352 (Prospective) | | 362 (Prospective) | |
| | Library Prep Method | | | | | | | |
| | Nextera | TruSeq | Nextera | TruSeq | Nextera | TruSeq | Nextera | TruSeq |
| | Flow cell | | | | | | | |
| | S1 | S2 | S1 | S2 | S1 | S2 | S1 | S2 |
| Raw Yield Per Flow | 389.9 | 945.4 | 381.8 | 946 | 365.3 | 869.9 | 398.3 | 440.7 |
| cell (Gb) | | | | | | | | |
| Reads Q >= 30 | 90.90% | 93.70% | 91.30% | 93.10% | 89.80% | 90.70% | 92.20% | 90.00% |
| % Cluster passing | 69.8/82.9 | 82.1/82.0 | 73.9/75.6 | 82.2/82.0 | 73.8/69.3 | 75.5/75.5 | 78.9/77.1 | 36.7/39.9 |
| filter, L1/L2 | | | | | | | | |
| % Error rate (ΦX174), | 0.19/0.42 | 0.27/0.47 | 0.25/0.65 | 0.27/0.37 | 0.25/0.45 | 0.31/0.37 | 0.20/0.36 | 0.33/0.41 |
| R1/R2 | | | | | | | | |
| Trimmed Yield (Gb) | 174.1 | 172.3 | 168.6 | 218.2 | 141 | 144.2 | 164.3 | 148.4 |
| Reads Mapped | 97.70% | 98.60% | 97.30% | 98.30% | 97.20% | 98.60% | 97.40% | 98.50% |
| Duplicate Reads | 11.50% | 6.50% | 11.60% | 7.30% | 8.90% | 9.20% | 9.90% | 3.90% |
| Mean Insert Size | 361.2 | 405.8 | 223.7 | 430 | 373.4 | 419.8 | 369 | 410 |
| (nt) | | | | | | | | |
| Average genome | 44.8 | 48.4 | 54 | 60.4 | 39.1 | 39.3 | 43.1 | 42.8 |
| coverage | | | | | | | | |
| % OMIM genes | 95.80% | 97.90% | 93.30% | 98.20% | 95.80% | 97.80% | 95.70% | 96.60% |
| w. >10X × 100% nt | | | | | | | | |
| Variants | 4,687,590 | 4,881,456 | 4,776,648 | 5,016,422 | 4,765,467 | 4,934,554 | 4,719,091 | 4,917,044 |
| Variants passing QC | 96.90% | 98.30% | 97.00% | 98.20% | 97.00% | 98.60% | 97.00% | 98.20% |
| CD Variants | 0.57% | 0.52% | 0.57% | 0.53% | 0.54% | 0.56% | 0.55% | 0.54% |
| Indels | 18.20% | 18.90% | 18.00% | 18.90% | 18.00% | 18.60% | 17.70% | 18.50% |
| Ti/Tv ratio | 2.02 | 2.02 | 2.03 | 2.03 | 2.02 | 2.03 | 2.02 | 2.01 |

| | Run | | | | | |
|---|---|---|---|---|---|---|
| | R18AB229 | Std | R18AB352 | Std | R18AB672 | Std |
| | Subject | | | | | |
| | 374 (Prospective) | | 7052 (Prospective) | | 412 (Prospective) | |
| | Library Prep Method | | | | | |
| | Nextera | KAPA | Nextera | NKAPA | Nextera | KAPA |
| | | Hyper | | Hyper | | Hyper |
| | Flow cell | | | | | |
| | S1 | S2 | S1 | S2 | S1 | S2 |
| Raw Yield Per Flow | 420.8 | 899.1 | 383.4 | 860.2 | 422.1 | 908.2 |
| cell (Gb) | | | | | | |
| Reads Q >= 30 | 93.30% | 91.60% | 90.10% | 90.10% | 92.90% | 91.60% |
| % Cluster passing | 83.0/81.8 | 78.3/77.8 | 75.49/74.7 | 75.2/74.1 | 83.1/82.3 | 78.9/78.8 |
| filter, L1/L2 | | | | | | |
| % Error rate (ΦX174), | 0.20/0.40 | 0.25/0.35 | 0.26/0.50 | 0.31/0.36 | 0.22/0.32 | 0.28/0.29 |
| R1/R2 | | | | | | |
| Trimmed Yield (Gb) | 185.5 | 267.8 | 156.4 | 138 | 183.4 | 203 |
| Reads Mapped | 98.00% | 98.50% | 97.30% | 98.30% | 98.60% | 98.60% |
| Duplicate Reads | 11.70% | 14.60% | 8.30% | 9.40% | 14.00% | 13.40% |
| Mean Insert Size | 266.9 | 423.8 | 371.4 | 428.4 | 338.1 | 416.2 |
| (nt) | | | | | | |
| Average genome | 48 | 68.4 | 41.6 | 37.3 | 47.6 | 50.9 |
| coverage | | | | | | |
| % OMIM genes | 96.00% | 98.40% | 95.20% | 97.80% | 96.90% | 98.20% |
| w. >10X × 100% nt | | | | | | |
| Variants | 4,758,713 | 5,001,708 | 4,821,433 | 4,981,748 | 4,958,194 | 4,965,915 |
| Variants passing QC | 98.10% | 98.00% | 98.10% | 98.60% | 98.10% | 98.20% |
| CD Variants | 0.55% | 0.53% | 0.56% | 0.53% | 0.56% | 0.53% |
| Indels | 19.60% | 18.80% | 17.60% | 18.50% | 18.70% | 18.90% |
| Ti/Tv ratio | 2.01 | 2.01 | 2.03 | 2.02 | 2.01 | 2.02 |

Abbreviations:
L: lane;
R: read;
nt: Nucleotides;
Gb: gigabase;
Q: Quality score;
OMIM: Online Mendelian Inheritance in Man;
QC: Quality Control;
CD: Coding Domain;
Ti/Tv ratio: ratio of the number of nucleotide transitions to the number of nucleotide transversions.

TABLE 4
Characteristics of sixteen children with genetic diseases used to train CNLP.

| | | | | | | | de |
|---|---|---|---|---|---|---|---|
| | | rWES or | | Affected | OMIM | | novo or |
| Family | S, D, T | rWGS | Disease | Gene | ID | Inheritance | inherited |
| 6007 | T | rWGS | EIEE9 | PCDH19 | 300088 | AD | DN |
| 6008 | S | rWGS | Glioblastoma | BRCA1 | 604370 | AD | n.d. |
| 6012 | S | rWGS | Coffin-Siris syndrome 1 | ARID1B | 135900 | AD | DN |
| 6014 | S | rWGS | Nemaline myopathy 2 | NEB | 256030 | AR | n.d. |
| 6024 | T | rWGS | Hypophosphatemic rickets, X- | PHEX | 307800 | XLD | I |
| | | | linked dominant | | | | |
| 6026 | T | rWGS | Alagille syndrome 1 | 20p12.2 del | 118450 | AD | DN |
| 6030 | T | rWGS | Neurofibromatosis 1; Left | NF1 & | 162200, | AD, | DN, I |
| | | | ventricular noncompaction 10 | MYBPC3 | 615396 | AD | |
| 6031 | T | rWGS | Catecholaminergic | RYR2 | 604772 | AD | DN |
| | | | polymorphicVentricular | | | | |
| | | | tachycardia 1 | | | | |
| 6037 | T | rWGS | Neonatal cholestasis; | none | none | n.a. | n.a. |
| | | | Extrahepatic biliary atresia | | | | |
| 6041 | T | rWGS | EIEE7 | KCNQ2 | 613720 | AD | DN |
| 6044 | S | rWGS | Pleuropulmonary blastoma | DICER | 601200 | AD | n.d. |
| 6045 | S | rWGS | Medulloblastoma | none | none | n.a. | n.a. |
| 6051 | S | rWGS | Glioma | none | none | n.a. | n.a. |
| 6052 | T | rWGS | MECRCN | TANGO2 | 616878 | AR | I |
| 6066 | D | rWGS | Neonatal cholestasis; Cleft lip | none | none | n.a. | n.a. |
| | | | and palate | | | | |
| 6117 | D | rWGS | Neonatal cholestasis | none | none | n.a. | n.a. |

| | | | V1 | V2 | Age at | | |
|---|---|---|---|---|---|---|---|
| | | | P/ | P/ | enrollment | | |
| Family | Variant 1 (V1) | Variant 2 (V2) | LP | LP | (days) | Sex | Consanguinity |
| 6007 | Xq22del | | | | 423 | F | No |
| 6008 | c.5159G > A, p.Arg1720Gln | | | | 4563 | F | No |
| 6012 | c.3096_3100delCAAAG; | | | | 231 | F | No |
| | p.Lys1033ArgfsTer32 | | | | | | |
| 6014 | c.19262 + 1G > A | c.2416-1G > C | | | 35 | M | No |
| 6024 | c.1604C > T, p.Thr535Met | | | | 137 | M | No |
| 6026 | Chr20: 10471400-13459331del | | | | 80 | M | U |
| 6030 | c.5118delT, | c.3184delG | LP | LP | 227 | M | No |
| | p.Val1707PhefsTer | p.Val1062LeufsTer13 | | | | | |
| 6031 | c.1646C > T; p.Ala549Val | | | | 6087 | F | No |
| 6037 | n.a. | | | | 60 | M | U |
| 6041 | c.875T > C; p.Leu292Pro | | | | 2 | F | No |
| 6044 | c.2771T > G; p.Leu924* | | | | 564 | M | U |
| 6045 | n.a. | | | | 5475 | M | U |
| 6051 | n.a. | | | | 2555 | M | U |
| 6052 | c.605 + 1G > A | 33 kb del TANGO | | | 898 | F | U |
| 6066 | n.a. | 2 exons 3-9 | | | 60 | F | U |
| 6117 | n.a. | | | | 60 | F | U |

Abbreviations:
EIEE: Early Infantile Epileptic Encephalopathy;
AD: Autosomal Dominant;
DN: de novo;
P: Pathogenic;
LP: Likely Pathogenic;
M: Male;
F: Female;
S: Singleton;
D: Duo;
T: Trio;
I: Inherited;
XLD: X-linked dominant;
MECRN: Metabolic encephalomyopathic crises, recurrent, with rhabdomyolysis, cardiac arrhythmias, and neurodegeneration;
U: undetermined;
OMIM: Online Mendelian Inheritance in Man.

TABLE 5
Precision and recall of phenotypic features extracted by CNLP from EhHRs in ten children with genetic diseases. Precision = tp/tp + fp.
Recall = tp/tp + fn.

| | | | | | | | de | |
|---|---|---|---|---|---|---|---|---|
| | | rWES | | | | | novo | |
| | S | or | | Affected | OMIM | | or | |
| Family | or T | rWGS | Disease | Gene | ID | Inheritance | inherited | Variant 1 (V1) |
| 201 | T | rWES | Prader Willi | 15q11-q13 | 176270 | AD | DN | Chr15: 23684685-26108259del |
| | | | Syndrome | del | | | | |
| 205 | T | rWGS | Dursun Syndrome | G6CP3 | 612541 | AR | I | c.207dupC, |
| | | | | | | | | p.Ile70HisfsTer17 |
| 213 | S | rWGS | Visceral Heterotaxy 5 | NODAL | 270100 | AD | I | c.778G > A, |
| | | | | | | | | p.Gly260Arg |
| 233 | T | rWGS | Tuberous Sclerosis 1 | TSC1 | 191100 | AD | DN | c.1498C > T, |
| | | | | | | | | p.Arg500Ter |
| 243 | T | rWGS | Pyridoxine dependent | ALDH7A1 | 266100 | AR | I | c.328C > T, |
| | | | seizures | | | | | p.Arg110Ter |
| 6094 | T | rWGS | Argininosuccinic | ASL | 207900 | AR | I | c.706C > T, |
| | | | Aciduria | | | | | p.Arg236Trp |
| 6098 | T | rWGS | Gaucher disease | GBA | 230800 | AR | I | c.1503C > G, |
| | | | | | | | | p.Asn501Lys |
| 6108 | T | rWGS | Tuberous Sclerosis 2 | TSC2 | 613254 | AD | DN | c.935_936delTC, |
| | | | | | | | | p.Leu312GlnfsTer25 |
| 7003 | T | rWGS | EIEE6 | SCN1A | 607208 | AD | DN | c.5555T > C, |
| | | | | | | | | p.Met1852Thr |
| 7004 | T | rWGS | Hypertrophic | MYH7 | 192600 | AD | I | c.746G > A, |
| | | | cardiomyopathy type 1 | | | | | p.Arg249Gln |
| Mean | | | | | | | | |
| Standard | | | | | | | | |
| Deviation | | | | | | | | |

| | | V1 | V2 | Age at | | | | | | OMIM CF |
|---|---|---|---|---|---|---|---|---|---|---|
| | | P/ | P/ | enrollment | | | CNLP | CNLP | CNLP | detected |
| Family | Variant 2 (V2) | LP | LP | (days) | Sex | Consanguinity | Features | Precision | Recall | by CNLP |
| 201 | | | | 3 | ♀ | U | 26 | 0.88 | n.d. | 3% |
| 205 | c.199_218 + 1delCTCAAC | P | P | 2 | ♂ | No | 96 | 0.80 | n.d. | 15% |
| | CTCATCTTCAAGTGG | | | | | | | | | |
| 213 | | | | 3 | ♂ | U | 95 | 0.67 | 0.91 | 56% |
| 233 | | | | 3 | ♀ | No | 158 | 0.51 | 0.91 | 14% |
| 243 | c.1279G > C, | | | 7 | ♂ | No | 85 | 0.82 | 0.93 | 21% |
| | p.Glu427Gln | | | | | | | | | |
| 6094 | c.706C > T, | P | P | 7 | ♀ | Yes | 90 | 0.83 | | 11% |
| | p.Arg236Trp | | | | | | | | | |
| 6098 | c.1448T > C, | | | 214 | ♀ | No | 96 | 0.9 | | 21% |
| | p.Leu483Pro | | | | | | | | | |
| 6108 | | | | 3 | ♂ | No | 83 | 0.76 | | 5% |
| 7003 | | | | 424 | ♂ | U | 44 | 0.84 | 0.93 | 25% |
| 7004 | | | | 5171 | ♂ | U | 71 | 0.94 | 0.96 | 44% |
| Mean | | | | | | | 86.7 | 0.80 | 0.93 | 22% |
| Standard | | | | | | | 32.8 | 0.13 | 0.02 | 0.17 |
| Deviation | | | | | | | | | | |

Abbreviations:
EIEE: Early Infantile Epileptic Encephalopathy;
AD: Autosomal Dominant;
AR: Autosomal Recessive;
DN: de novo;
P: Pathogenic;
LP: Likely Pathogenic;
S: Singleton;
T: Trio;
I: Inherited;
U: undetermined;
OMIM: Online Mendelian Inheritance in Man;
CF: Clinical Feature.

TABLE 6
Number of structural variants shortlisted by MOON and
rank of the causal variant in MOON in 11 children with
genetic diseases. All samples were run as singletons.

| | | # SV | # SV | Causal SV |
|---|---|---|---|---|
| | rWES/ | calls in | shortlisted by | rank in |
| Family | rWGS | gVCF | MOON | MOON |
| 201 | rWES | 6 | 2 | 1 |
| 259 | rWES | 16 | 9 | 1 |
| 286 | rWES | 7 | 3 | 1 |
| 319 | rWES | 12 | 4 | 1 |
| 217 | rWGS | 21 | 8 | 1 |
| 223 | rWGS | 16 | 9 | 5 |
| 302 | rWGS | 22 | 17 | 13 |
| 6140 | rWGS | 11 | 8 | 1 |
| 6146 | rWGS | 23 | 15 | 9 |
| 6164 | rWGS | 25 | 15 | 12 |
| 7023 | rWGS | 17 | 12 | 12 |
| Mean, rWES | | 10.3 | 4.5 | Median |
| | | | | rWGS, rWES |
| Mean, rWGS | | 19.3 | 12.0 | 1.0 |

Abbreviations:
gVCF: Genomic variant call file;
rWES: rapid whole exome sequencing;
rWGS: rapid whole genome sequencing;
SV: structural variant.

TABLE 7
Summary statistics of provisional diagnoses reported for rapid clinical
genome sequencing. Total probands refers to children tested.

| | | Mean Time to Provisional Report |
|---|---|---|
| | Provisional | (Sample Accession to Preliminary |
| Total Probands | Reports Returned | Results Communicated), Days |
| 684 | 114 (16.7%) | 3.6 |

Although the invention has been described with reference to the above examples, it will be understood that modifications and variations are encompassed within the spirit and scope of the invention. Accordingly, the invention is limited only by the following claims.

What is claimed is:

1. A method comprising:
a) determining a phenome of a subject from an electronic medical record (EMR), wherein the phenome comprises a plurality of clinical phenotypes extracted from the EMR;
b) translating the clinical phenotypes into standardized vocabulary;
c) generating a first list of potential differential diagnoses of the subject;
d) performing genetic sequencing of a DNA sample from the subject;
e) determining genetic variants of the DNA;
f) analyzing the results of (c) and (e) to generate a second list of potential differential diagnoses of the subject, the second list being rank ordered; and
g) generating a report comprising results of the analysis of (f).

2. The method of claim 1, further comprising generating the EMR for the subject prior to (a).

3. The method of claim 1, wherein (b) utilizes natural language processing to perform the translation.

4. The method of claim 1, wherein (a)-(c) and (d)-(e) are performed in parallel.

5. The method of claim 1, wherein genetic sequencing comprises rapid whole genome sequencing (rWGS), ultra-rapid whole genome sequencing, or rapid whole exome sequencing (rWES).

6. The method of claim 5, wherein the DNA sample is from a biological sample.

7. The method of claim 6, wherein the sample is serum, saliva, buccal smear/swab, plasma, feces, cerebrospinal fluid or urine.

8. The method of claim 6, wherein the sample is blood.

9. The method of claim 6, wherein the biological sample is a dried blood spot.

10. The method of claim 5, wherein genetic sequence comprises rWGS and rWES.

11. The method of claim 1, wherein the ranked list is performed via query of a database populated with known clinical phenotypes expressed in the same vocabulary as the standardized vocabulary of (b).

12. The method of claim 1, wherein determining genetic variants of (f) further comprises annotation and classification of the genetic variants.

13. The method of claim 12, wherein the genetic variants are utilized to generate a probabilistic diagnosis.

14. The method of claim 12, wherein the genetic variants are annotated and classified as being of uncertain significance (VUS), pathogenic (P) or likely pathogenic (LP).

15. The method of claim 12, wherein only genetic variants with an allele frequency of <5%, 2.5%, 1%, 0.1% or less in a population of healthy individuals is retained.

16. The method of claim 15, wherein determining genetic variants of (e) further comprises annotation of the genetic variants to identify and rank all diplotypes as being of uncertain significance (VUS), pathogenic (P) or likely pathogenic (LP) on the basis of pathogenicity.

17. The method of claim 16, wherein the second list of potential differential diagnoses is generated by comparing the annotated VUS, LP and P diplotypes on a regional genomic basis with corresponding genomic regions associated with the first list of potential differential diagnoses of (c).

18. The method of claim 17, wherein the genetic variants are ranked based on a combination of rank of goodness of fit of clinical phenotypes, rank of pathogenicity of diplotypes, and/or allele frequencies of the genetic variants in a population of health individuals.

19. The method of claim 1, further comprising performing genetic sequencing of a DNA sample from a biological parent of the subject.

20. The method of claim 19, wherein genetic sequencing is performed for both biological parents and only results in which trio diplotypes fit a known inheritance pattern of a specific genetic disease are obtained.

21. The method of claim 19, wherein genetic sequencing is performed for both biological parents, wherein parental health status (healthy or affected) is used to obtain only results in which parental diplotypes fit a known inheritance pattern of a specific genetic disease.

22. The method of claim 19, wherein genetic variants present in the subject's genome and not in the parental genome are utilized to determine a diagnosis for the subject.

23. The method of claim 1, wherein genetic sequence comprises sequencing of a whole genome, whole exome, or gene panel.

24. The method of claim 1, wherein the subject is less than 5 years old.

25. The method of claim 24, wherein the subject is an infant, fetus or neonate.

26. The method of claim 1, wherein the potential differential diagnoses comprise genetic diseases.

27. The method of claim 1, wherein the method is automated.

28. The method of claim 1, further comprising generating a therapy regime for the subject based on (g).

29. The method of claim 1, further comprising providing a therapy to the subject.

30. The method of claim 1, wherein (a) further comprises analyzing supplemental clinical information to determine the phenome.

31. The method of claim 1, wherein (a) is performed for a plurality of subjects thereby generating a plurality of EMRs, a plurality of phenomes, and a plurality of clinical phenotypes.

32. The method of claim 2, wherein (a) is performed for a plurality of subjects thereby generating a plurality of EMRs, a plurality of phenomes, and a plurality of clinical phenotypes.

33. The method of claim 31, further comprising storing on a non-transitory memory the plurality of EMRs, the plurality of phenomes, and the plurality of clinical phenotypes to generate a searchable database.

34. The method of claim 32, further comprising storing on a non-transitory memory the plurality of EMRs, the plurality of phenomes, and the plurality of clinical phenotypes to generate a searchable database.

35. The method of claim 33, further comprising utilizing the database to screen for genetic data, a genotype, or a disease or disorder in a second subject or to update a diagnosis of the subject.

36. The method of claim 34, further comprising utilizing the database to screen for genetic data, a genotype, or a disease or disorder in a second subject or to update a diagnosis of the subject.

37. A system comprising:
a controller including at least one processor and non-transitory memory, wherein the controller is configured to perform (a)-(c) and (e)-(g) of claim 1.

38. A method comprising:
a) generating a plurality of electronic medical records (EMRs) for a plurality of subjects;
b) determining a plurality of phenomes of the plurality of subjects from the EMRs using natural language processing, wherein the phenomes each comprise a plurality of clinical phenotypes extracted from each of the EMRs; and
c) storing on a non-transitory memory the plurality of EMRs, the plurality of phenomes, and the plurality of clinical phenotypes to generate a searchable database;
d) utilizing the database to screen for a disease or disorder in a new subject or to update a diagnosis of one of the plurality of subjects.

39. A system comprising:
a controller including at least one processor and non-transitory memory, wherein the controller is configured to perform (a)-(c) of claim 38.
